(19) United States
(12) Patent Application Publication
(10) Pub. No.: US 2026/0294347 A1
(43) Pub. Date: Oct. 1, 2026

(54) THIN-FILM LEAD ASSEMBLIES AND NEURAL INTERFACES

(71) Applicant: Verily Life Sciences LLC, Dallas, TX (US)

(72) Inventors: Kedar Shah, San Francisco, CA (US); Annapurna Karicherla, South San Francisco, CA (US); Cindy Au, South San Francisco, CA (US)

(73) Assignee: Verily Life Sciences LLC, Dallas, TX (US)

(21) Appl. No.: 19/571,799

(22) Filed: Mar. 19, 2026

Related U.S. Application Data
(63) Continuation of application No. 17/441,597, filed on Mar. 23, 2020.
(60) Provisional application No. 62/822,189, filed on Mar. 22, 2019.

(51) Int. Cl. A61B 5/00; A61N 1/05; H10K 10/46; H10K 85/00; H10K 85/10

(52) U.S. Cl. CPC A61B 5/6868; A61N 1/0529; A61N 1/0551; H10K 10/471; H10K 85/1135; H10K 85/731; A61B 2562/0209

(57) ABSTRACT

The present disclosure relates to thin-film lead assemblies and neural interfaces, and methods of microfabricating thin-film lead assemblies and neural interfaces. Particularly, aspects of the present disclosure are directed to a thin-film neural interface that includes a proximal end, a distal end, a supporting structure that extends from the proximal end to the distal end, one or more of conductive traces formed on a portion of the supporting structure, one or more electrodes formed on the front side of the supporting structure in electrical connection with the one or more conductive traces, and a backing formed on the back side of the supporting structure. The supporting structure comprises one or more features to facilitate mechanical adhesion between the supporting structure and the backing.

### CROSS-REFERENCE TO RELATED APPLICATIONS

The present application is a continuation of U.S. patent application Ser. No. 17/441,597, filed Sep. 21, 2021, which is a national-phase application under 35 U.S.C. 371 of International Patent Application No. PCT/US2020/024173, filed Mar. 23, 2020, which claims priority and benefit from U.S. Provisional Patent Application No. 62/822,189, filed Mar. 22, 2019, the disclosures of which are incorporated by reference herein in their entireties.

### FIELD OF THE INVENTION

The present disclosure relates to implantable neuromodulation devices and methods of fabrication, and in particular to thin-film lead assemblies and neural interfaces, and methods of microfabricating thin-film lead assemblies and neural interfaces.

### BACKGROUND

Normal neural activity is an intricate balance of electrical and chemical signals, which can be disrupted by a variety of insults (genetic, chemical or physical trauma) to the nervous system, causing cognitive, motor and sensory impairments. Similar to the way a cardiac pacemaker or defibrillator corrects heartbeat abnormalities, neuromodulation therapies help to reestablish normal neural balance. In particular instances, neuromodulation therapies utilize medical device technologies to enhance or suppress activity of the nervous system for the treatment of disease. These technologies include implantable as well as non-implantable neuromodulation devices and systems that deliver electrical, chemical or other agents to reversibly modify brain and nerve cell activity. The most common neuromodulation therapy is spinal cord stimulation to treat chronic neuropathic pain. In addition to chronic pain relief, some examples of neuromodulation therapies include deep brain stimulation for essential tremor, Parkinson's disease, dystonia, epilepsy and psychiatric disorders such as depression, obsessive compulsive disorder and Tourette syndrome; sacral nerve stimulation for pelvic disorders and incontinence; vagus nerve stimulation for rheumatoid arthritis; gastric and colonic stimulation for gastrointestinal disorders such as dysmotility or obesity; vagus nerve stimulation for epilepsy, obesity or depression; carotid artery stimulation for hypertension, and spinal cord stimulation for ischemic disorders such as angina and peripheral vascular disease.

Neuromodulation devices and systems tend to have a similar form factor, derived from their predecessors, e.g. the pacemaker or defibrillator. Such neuromodulation devices and systems typically consist of an implant comprising a neurostimulator having electronics connected to a lead assembly that delivers electrical pulses to electrodes interfaced with nerves or nerve bundles via a neural interface. The lead assembly is typically formed of a conductive material and takes the form of an insulated wire connected to the neural interface via a first connector on one end (e.g., a distal end) and the electronics of the neurostimulator via a second connector on another end (e.g., a proximal end). In some instances (e.g., deep implants), the lead assembly comprises additional conductors and connectors such as extension wires or a cable connected via connectors between the electrodes and the electronics of the neurostimulator.

Conventional microfabrication processes enable neural interfaces of significant complexity such as retinal prostheses. For example, neural interfaces formed from flexible electronics may be manufactured using lithographic patterning and lamination methods that enable smaller feature sizes and increased scalability. Flexible electronics, also known as flex circuits, is a technology for assembling electronic circuits by mounting electronic devices on flexible substrates, such as polyimide, polyether ether ketone (PEEK), or transparent conductive polyester film. Most flexible substrates used for microfabricated neural interfaces maintain some rigidity, and thus are mechanically mismatched with the neural tissue. As a result, the flexible substrates may be overmolded with softer materials such as silicones and urethanes in order to mechanically match with the neural tissue. However, adhesion of the softer materials to flexible substrates can degrade over time, exposing the flexible substrate to the tissue. This loss of adhesion can eventually result in release of the flexible substrates from the soft material backing. In view of these factors, it may be desirable to develop neuromodulation devices and systems with neural interfaces that are capable of having design flexibility such that the electrodes sit as close to the neural tissue as possible, and desirable mechanical properties to mitigate loss of adhesion of the softer materials and improve upon reliability of performance.

### BRIEF SUMMARY

In various embodiments, a thin-film neural interface is provided comprising: a supporting structure comprised of one or more layers of dielectric material, where the supporting structure comprises a front side and a back side; one or more conductive traces formed on the one or more layers of dielectric material; one or more electrodes formed on the front side of the supporting structure in electrical connection with the one or more conductive traces; and a backing formed on the back side of the supporting structure, where the backing is comprised of a medical grade polymer material, the supporting structure includes one or more through holes, and the medical grade polymer material fills at least a portion of each of the one or more through holes.

In some embodiments, the one or more layers of dielectric material have a thickness from 1 μm to 100 μm, and the dielectric material is polyimide, liquid crystal polymer, parylene, polyether ether ketone, or a combination thereof.

In some embodiments, the one or more conductive traces have a thickness from 0.05 μm to 25 μm, the one or more conductive traces are comprised of one or more layers of conductive material, and the conductive material is gold (Au), gold/chromium (Au/Cr), platinum (Pt), platinum/iridium (Pt/Ir), titanium (Ti), gold/titanium (Au/Ti), or any alloy thereof.

In some embodiments, the one or more electrodes have a thickness from 0.05 μm to 25 μm, the one or more electrodes are comprised of one or more layers of conductive material, and the conductive material is PEDOT (Poly(3,4-ethylenedioxythiophene)), gold (Au), gold/chromium (Au/Cr), platinum (Pt), platinum/iridium (Pt/Ir), titanium (Ti), gold/titanium (Au/Ti), or any alloy thereof.

In some embodiments, the backing has a thickness from 10 μm to 150 μm, and the medical grade polymer material is silicone, a polymer dispersion, parylene, or a polyurethane.

In some embodiments, the supporting structure further comprises opposing edges, the backing wraps around the opposing edges from the back side of the supporting structure, and the backing is coplanar with the front side of the supporting structure.

In other embodiments, the supporting structure further comprises opposing edges, and the backing wraps around the opposing edges from the back side of the supporting structure.

Optionally, the opposing edges comprise a pattern in the one or more layers of dielectric material, and the backing further comprises a pattern in the medical grade polymer material that interlocks with the pattern in the one or more layers of dielectric material. Optionally, the backing is overmolded over the opposing edges, and the medical grade polymer material forms a backing layer on a portion of the front side of the supporting structure that is adjacent to the opposing edges. In some embodiments, the backing layer has a thickness from 10 μm to 150 μm. In some embodiments, the opposing edges are extended or folded to maintain a predetermined distance between the backing layer and the one or more electrodes. Optionally, the predetermined distance is from 0.25 mm to 25 mm.

In various embodiments, a thin-film neural interface is provided comprising: a supporting structure comprised of one or more layers of dielectric material, where the supporting structure comprises a front side, a back side, and opposing edges; one or more conductive traces formed on the one or more layers of dielectric material; one or more electrodes formed on the front side of the supporting structure in electrical connection with the one or more conductive traces; and a backing formed on the back side of the supporting structure and wraps around the opposing edges from the back side of the supporting structure, where the backing is comprised of a medical grade polymer material, the opposing edges comprise a pattern in the one or more layers of dielectric material, and the backing further comprises a pattern in the medical grade polymer material that interlocks with the pattern in the one or more layers of dielectric material.

In some embodiments, the one or more layers of dielectric material have a thickness from 1 μm to 100 μm, and the dielectric material is polyimide, liquid crystal polymer, parylene, polyether ether ketone, or a combination thereof.

In some embodiments, the one or more conductive traces have a thickness from 0.05 μm to 25 μm, the one or more conductive traces are comprised of one or more layers of conductive material, and the conductive material is gold (Au), gold/chromium (Au/Cr), platinum (Pt), platinum/iridium (Pt/Ir), titanium (Ti), gold/titanium (Au/Ti), or any alloy thereof.

In some embodiments, the one or more electrodes have a thickness from 0.05 μm to 25 μm, the one or more electrodes are comprised of one or more layers of conductive material, and the conductive material is PEDOT (Poly(3,4-ethylenedioxythiophene)), gold (Au), gold/chromium (Au/Cr), platinum (Pt), platinum/iridium (Pt/Ir), titanium (Ti), gold/titanium (Au/Ti), or any alloy thereof.

In some embodiments, the backing has a thickness from 10 μm to 150 μm, and the medical grade polymer material is silicone, a polymer dispersion, parylene, or a polyurethane.

In some embodiments, the backing is coplanar with the front side of the supporting structure.

In various embodiments, a thin-film neural interface is provided comprising: a supporting structure comprised of one or more layers of dielectric material, where the supporting structure comprises a front side, a back side, and opposing edges; one or more conductive traces formed on the one or more layers of dielectric material; one or more electrodes formed on the front side of the supporting structure in electrical connection with the one or more conductive traces; and a backing formed on the back side of the supporting structure and wraps around the opposing edges from the back side of the supporting structure, where the backing is comprised of a medical grade polymer material, the backing is overmolded over the opposing edges, and the medical grade polymer material forms a backing layer on a portion of the front side of the supporting structure that is adjacent to the opposing edges.

In some embodiments, the one or more layers of dielectric material have a thickness from 1 μm to 100 μm, and the dielectric material is polyimide, liquid crystal polymer, parylene, polyether ether ketone, or a combination thereof.

In some embodiments, the one or more conductive traces have a thickness from 0.05 μm to 25 μm, the one or more conductive traces are comprised of one or more layers of conductive material, and the conductive material is gold (Au), gold/chromium (Au/Cr), platinum (Pt), platinum/iridium (Pt/Ir), titanium (Ti), gold/titanium (Au/Ti), or any alloy thereof.

In some embodiments, the one or more electrodes have a thickness from 0.05 μm to 25 μm, the one or more electrodes are comprised of one or more layers of conductive material, and the conductive material is PEDOT (Poly(3,4-ethylenedioxythiophene)), gold (Au), gold/chromium (Au/Cr), platinum (Pt), platinum/iridium (Pt/Ir), titanium (Ti), gold/titanium (Au/Ti), or any alloy thereof.

In some embodiments, the backing has a thickness from 10 μm to 150 μm, and the medical grade polymer material is silicone, a polymer dispersion, parylene, or a polyurethane.

In some embodiments, the backing layer has a thickness from 10 μm to 150 μm.

In some embodiments, the opposing edges are extended or folded to maintain a predetermined distance between the backing layer and the one or more electrodes. Optionally, the predetermined distance is from 0.25 mm to 25 mm.

In various embodiments, a thin-film lead assembly is provided comprising: a cable comprising a supporting structure and a plurality of conductive traces formed on a portion of the supporting structure, where the supporting structure is comprised of one or more layers of dielectric material; an thin-film neural interface formed on the supporting structure at a distal end of the cable, where the thin-film neural interface comprises: (i) one or more electrodes formed on a front side of the supporting structure in electrical connection with one or more conductive traces of the plurality of conductive traces, and (ii) a backing formed on a back side of the supporting structure, where the backing is comprised of a medical grade polymer material, and the supporting structure includes one or more features for mechanical adhesion with the backing; and a connector in electrical connection with the one or more conductive traces of the plurality of conductive traces at a proximal end of the cable.

In some embodiments, the supporting structure further comprises opposing edges, the backing wraps around the opposing edges from the back side of the supporting structure, and the backing is coplanar with the front side of the supporting structure.

In some embodiments, the supporting structure further comprises opposing edges, and the backing wraps around the opposing edges from the back side of the supporting structure.

In some embodiments, the one or more features comprise one or more through holes, and the medical grade polymer material fills at least a portion of each of the one or more through holes.

In some embodiments, the one or more features comprise a pattern formed in the one or more layers of dielectric material at the opposing edges, and the backing further comprises a pattern in the medical grade polymer material that interlocks with the pattern in the one or more layers of dielectric material.

In some embodiments, the backing is overmolded over the opposing edges, the medical grade polymer material forms a backing layer on a portion of the front side of the supporting structure that is adjacent to the opposing edges, and the one or more features comprise the opposing edges being extended or folded to maintain a predetermined distance between the backing layer and the one or more electrodes.

In various embodiments, a method of manufacturing a thin-film neural interface is provided that comprises: obtaining an initial structure comprising: a proximal end, a distal end, a supporting structure that extends from the proximal end to the distal end, one or more of conductive traces formed on a portion of the supporting structure, and one or more electrodes in electrical connection with the one or more conductive traces of the plurality of conductive traces, where the one or more electrodes are formed on a front side of the supporting structure, and the supporting structure comprises one or more features for mechanical adhesion with a backing; adding a manipulation device to the initial structure, where the manipulation device extends from the proximal end to the distal end of the initial structure, and the manipulation device hangs over each of the proximal end and the distal end; attaching, using the manipulation device, the initial structure to a mandrel; loading the mandrel with the attached initial structure into a cavity of a mold; injecting a backing material into the cavity of the mold to form a backing over a back side of the supporting structure; heating the backing and the initial structure attached to the mandrel to form the thin-film neural interface with the backing attached to the back side of the supporting structure via the one or more features; and removing the mandrel from the thin-film neural interface.

In various embodiments, a method of manufacturing a thin-film neural interface is provided that comprises: obtaining an initial structure comprising: a proximal end, a distal end, a supporting structure that extends from the proximal end to the distal end, one or more of conductive traces formed on a portion of the supporting structure, and one or more electrodes in electrical connection with the one or more conductive traces of the plurality of conductive traces, where the one or more electrodes are formed on a front side of the supporting structure, and the supporting structure comprises one or more features for mechanical adhesion with a backing; adding a manipulation device to the initial structure, where the manipulation device extends from the proximal end to the distal end of the initial structure, and the manipulation device hangs over each of the proximal end and the distal end; attaching, using the manipulation device, the initial structure to a mandrel; inserting the mandrel with the attached initial structure into a tube of backing material to form an intermediate structure; heating the intermediate structure to reflow the tube of backing material and form the thin-film neural interface with the backing attached to a back side of the supporting structure via the one or more features; and removing the mandrel from the thin-film neural interface.

In some embodiments, the obtaining the initial structure comprises: forming a first polymer layer of the supporting structure on a wafer or panel of substrate; forming the one or more conductive traces on a first portion of the first polymer layer; forming a wiring layer on a second portion of the first polymer layer, where the forming the wiring layer comprises depositing a conductive material in electrical contact with the one or more of conductive traces; depositing a second polymer layer of the supporting structure on the wiring layer and the second portion of the first polymer layer; forming the one or more electrodes on the second polymer layer such that the one or more electrodes are in electrical contact with at least a portion of a top surface of the wiring layer; forming the one or more features in the first polymer layer, the second polymer layer, or a combination thereof; cutting the initial structure from the first polymer layer and the second polymer layer; and removing the initial structure from the wafer or panel of substrate.

In some embodiments, the supporting structure is comprised of one or more layers of dielectric material.

In some embodiments, the one or more layers of dielectric material have a thickness from 1 μm to 100 μm, and the dielectric material is polyimide, liquid crystal polymer, parylene, polyether ether ketone, or a combination thereof.

In some embodiments, the one or more conductive traces have a thickness from 0.05 μm to 25 μm, the one or more conductive traces are comprised of one or more layers of conductive material, and the conductive material is gold (Au), gold/chromium (Au/Cr), platinum (Pt), platinum/iridium (Pt/Ir), titanium (Ti), gold/titanium (Au/Ti), or any alloy thereof.

In some embodiments, the one or more electrodes have a thickness from 0.05 μm to 25 μm, the one or more electrodes are comprised of one or more layers of conductive material, and the conductive material is PEDOT (Poly(3,4-ethylenedioxythiophene)), gold (Au), gold/chromium (Au/Cr), platinum (Pt), platinum/iridium (Pt/Ir), titanium (Ti), gold/titanium (Au/Ti), or any alloy thereof.

In some embodiments, the backing has a thickness from 10 μm to 150 μm, and the backing material is silicone, a polymer dispersion, parylene, or a polyurethane.

In some embodiments, the supporting structure comprises opposing edges, and where the mold, the mandrel, or a combination thereof comprises a design feature such that when the backing material is injected or reflowed the backing is formed wrapping around the opposing edges from the back side of the supporting structure, and the backing is coplanar with the front side of the supporting structure.

In some embodiments, the supporting structure comprises opposing edges, and where the mold, the mandrel, or a combination thereof comprises a first design feature such that when the backing material is injected or reflowed the backing is formed wrapping around the opposing edges from the back side of the supporting structure.

In some embodiments, the one or more features comprise one or more through holes, and the backing material fills at least a portion of each of the one or more through holes.

In some embodiments, the one or more features comprise a pattern formed in the supporting structure at the opposing edges, and where the mold, the mandrel, or a combination thereof comprises a second design feature such that when the backing material is injected or reflowed the backing is formed comprising a pattern that interlocks with the pattern in the supporting structure.

In some embodiments, the mold, the mandrel, or a combination thereof comprises a third design feature such that when the backing material is injected or reflowed the backing is overmolded over the opposing edges, the backing material forms a backing layer on a portion of the front side of the supporting structure that is adjacent to the opposing edges, and the one or more features comprise the opposing edges being extended or folded to maintain a predetermined distance between the backing layer and the one or more electrodes.

### BRIEF DESCRIPTION OF THE DRAWINGS

The present invention will be better understood in view of the following non-limiting figures, in which:

FIG. 1 shows a neuromodulation system in accordance with various embodiments;

FIGS. 2A, 2B, and 2C show a thin-film neural interface in accordance with various embodiments;

FIGS. 3A and 3B show an alternative thin-film neural interface in accordance with various embodiments;

FIGS. 4A and 4B show an alternative thin-film neural interface in accordance with various embodiments;

FIGS. 5A, 5B, and 5C show an alternative thin-film neural interface in accordance with various embodiments;

FIGS. 6A, 6B, 6C, 6D, 6E, and 6F show thin-film neural interface views illustrating a method of forming a thin-film neural interface in accordance with various embodiments;

FIGS. 7A, 7B, 7C, 7D, 7E, 7F, 7G, and 7H show thin-film neural interface views illustrating a method of forming a thin-film neural interface in accordance with various embodiments; and FIGS. 8A, 8B, 8C, 8D, 8E, 8F, 8G, and 8H show thin-film neural interface views illustrating an alternative method of forming a thin-film neural interface in accordance with various embodiments.

### DETAILED DESCRIPTION

### I. Introduction

The following disclosure describes thin-film lead assemblies and neural interfaces, and methods of microfabricating thin-film lead assemblies and neural interfaces. As used herein, the phrases “microfabrication” and “microfabricating” refers to the process of fabricating miniature structures on micrometer scales and smaller. The major concepts and principles of microfabrication are microlithography, doping, thin-films, etching, bonding, and polishing. As used herein, the phrase “thin-films” refers to a layer of material ranging from fractions of a nanometer (monolayer) to several micrometers in thickness (e.g., between a few nanometers to about 100 μm, or the thickness of a few atoms). Thin-films may be deposited by applying a very thin film of material (e.g., between a few nanometers to about 100 μm, or the thickness of a few atoms) onto a substrate surface to be coated, or onto a previously deposited layer of thin film. In various embodiments, the thin-film lead assemblies and neural interfaces are provided comprising a substrate (e.g., a flexible substrate or supporting structure), one or more electrodes formed on a front side of the base polymer layer, and a backing formed on the back side of the base polymer layer.

Limitations associated with conventional thin-film lead assemblies and neural interfaces is that the substrate (e.g., a flexible substrate or supporting structure) used for the interface maintains some rigidity, and thus is mechanically mismatched with the neural tissue. As a result, the substrate may be overmolded with softer materials such as silicones and urethanes in order to mechanically match with the neural tissue. The softer materials may be spin coated or overmolded to the back side of an unperforated substrate. There is a desire to only use the softer materials as the backing on the back side of the substrate (e.g., without an intervening adhesion layer) so that the electrodes on the front side of the substrate can sit as close to the neural tissue as possible. However, there are challenges to creating such a neural interface without poor mechanical and reliability performance. For example, adhesion of the softer materials to substrates can degrade over time under exposure to bodily fluid, exposing the substrate to the tissue. This loss of adhesion can eventually result in release of the substrates from the soft material backing and ultimately results in mechanical and/or performance failure.

To address these limitations and problems, the thin-film neural interface of various embodiments disclosed herein comprises a supporting structure that has one or more features structured to facilitate mechanical adhesion between the supporting structure and the backing.

The one or more features may include: (i) through holes in the supporting structure, (ii) a pattern formed in the supporting structure, (iii) edges of the supporting structure that are extended or folded, or (iv) any combination thereof. One illustrative embodiment of the present disclosure is directed to a thin-film neural interface comprising: a supporting structure comprised of one or more layers of dielectric material, where the supporting structure comprises a front side and a back side; one or more conductive traces formed on the one or more layers of dielectric material; one or more electrodes formed on the front side of the supporting structure in electrical connection with the one or more conductive traces; and a backing formed on the back side of the supporting structure. The backing is comprised of a medical grade polymer material, the supporting structure includes one or more through holes, and the medical grade polymer material fills at least a portion of each of the one or more through holes.

In other embodiments, a thin-film neural interface is provided comprising: a supporting structure comprised of one or more layers of dielectric material, where the supporting structure comprises a front side, a back side, and opposing edges; one or more conductive traces formed on the one or more layers of dielectric material; one or more electrodes formed on the front side of the supporting structure in electrical connection with the one or more conductive traces; and a backing formed on the back side of the supporting structure and wraps around the opposing edges from the back side of the supporting structure. The backing is comprised of a medical grade polymer material, the opposing edges comprise a pattern in the one or more layers of dielectric material, and the backing further comprises a pattern in the medical grade polymer material that interlocks with the pattern in the one or more layers of dielectric material.

In other embodiments, a thin-film neural interface is provided comprising: a supporting structure comprised of one or more layers of dielectric material, where the supporting structure comprises a front side, a back side, and opposing edges; one or more conductive traces formed on the one or more layers of dielectric material; one or more electrodes formed on the front side of the supporting structure in electrical connection with the one or more conductive traces; and a backing formed on the back side of the supporting structure and wraps around the opposing edges from the back side of the supporting structure. The backing is comprised of a medical grade polymer material, the backing is overmolded over the opposing edges, and the medical grade polymer material forms a backing layer on a portion of the front side of the supporting structure that is adjacent to the opposing edges.

In other embodiments, a thin-film lead assembly is provided comprising: a cable comprising a supporting structure and a plurality of conductive traces formed on a portion of the supporting structure, where the supporting structure is comprised of one or more layers of dielectric material; an thin-film neural interface formed on the supporting structure at a distal end of the cable, where the thin-film neural interface comprises: (i) one or more electrodes formed on a front side of the supporting structure in electrical connection with one or more conductive traces of the plurality of conductive traces, and (ii) a backing formed on a back side of the supporting structure, and where the backing is comprised of a medical grade polymer material, and the supporting structure includes one or more features for mechanical adhesion with the backing; and a connector in electrical connection with the one or more conductive traces of the plurality of conductive traces at a proximal end of the cable.

To further address these limitations and problems, a method of manufacturing a thin-film neural interface of various embodiments disclosed herein includes process steps for creating a structure, which results in improved mechanical adhesion between the supporting structure and the backing, a smaller footprint, and greater design flexibility. One illustrative embodiment of the present disclosure is directed to method of manufacturing a thin-film neural interface that comprises obtaining an initial structure comprising: a proximal end, a distal end, a supporting structure that extends from the proximal end to the distal end, one or more of conductive traces formed on a portion of the supporting structure, and one or more electrodes in electrical connection with the one or more conductive traces of the plurality of conductive traces, where the one or more electrodes are formed on a front side of the supporting structure, and the supporting structure comprises one or more features for mechanical adhesion with a backing; adding a suture to the initial structure, where the suture extends from the proximal end to the distal end of the initial structure, and the suture hangs over each of the proximal end and the distal end; attaching, using the suture, the initial structure to a mandrel; loading the mandrel with the attached initial structure into a cavity of a mold; injecting a backing material into the cavity of the mold to form the backing over a back side of the supporting structure; heating the backing and the initial structure attached to the mandrel to form the thin-film neural interface with the backing attached to the back side of the supporting structure via the one or more features; and removing the mandrel from the thin-film neural interface.

In other embodiments, a method of manufacturing a thin-film neural interface is provided that comprises obtaining an initial structure comprising: a proximal end, a distal end, a supporting structure that extends from the proximal end to the distal end, one or more of conductive traces formed on a portion of the supporting structure, and one or more electrodes in electrical connection with the one or more conductive traces of the plurality of conductive traces, where the one or more electrodes are formed on a front side of the supporting structure, and the supporting structure comprises one or more features for mechanical adhesion with a backing; adding a suture to the initial structure, where the suture extends from the proximal end to the distal end of the initial structure, and the suture hangs over each of the proximal end and the distal end; attaching, using the suture, the initial structure to a mandrel; inserting the mandrel with the attached initial structure into a tube of backing material to form an intermediate structure; heating the intermediate structure to reflow the tube of backing material and form the thin-film neural interface with the backing attached to a back side of the supporting structure via the one or more features; and removing the mandrel from the thin-film neural interface.

Advantageously, these approaches provide a thin-film neural interface, which has improved mechanical adhesion between the supporting structure and the backing, a smaller footprint, and greater design flexibility. This solution is scalable to interface multiple electrodes with tissue using thin film substrates, and thus enabling several therapeutic opportunities for neurostimulation. Furthermore even for applications where multiple electrodes are not required, various embodiments can be miniaturized to make the implant minimally invasive, additionally may make invasive anatomies to become accessible (or navigable) due to the miniaturization. It should be understood that although deep brain neurostimulation and vagus nerve or artery/nerve plexus device applications are provided as examples of some embodiments, this solution is applicable to all interfaces, leads, and devices that need electrodes/sensors interfaced with tissue.

### II. Neuromodulation Devices and Systems With a Thin-Film Neural Interface

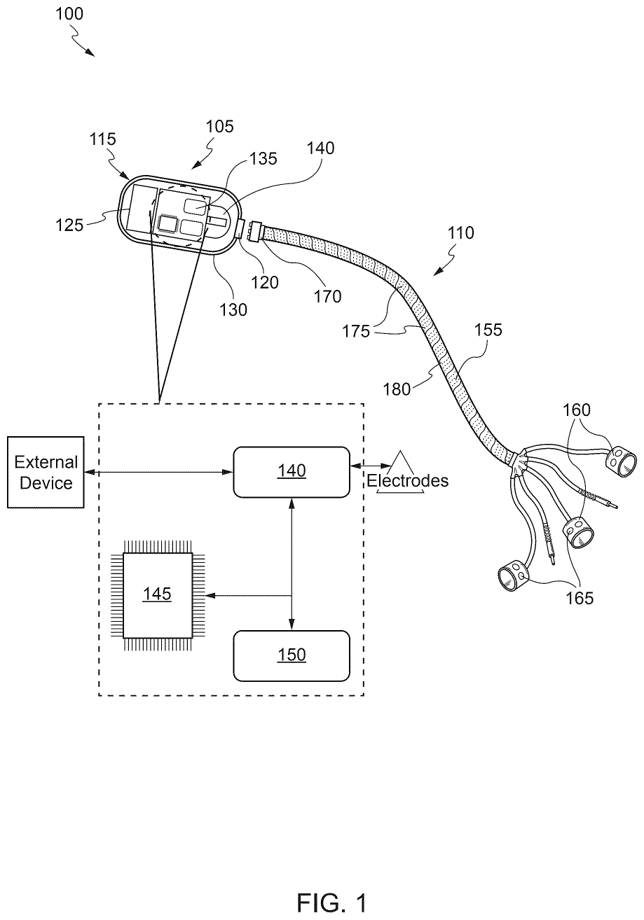

FIG. 1 shows a neuromodulation system 100 in accordance with some aspects of the present invention. In various embodiments, the neuromodulation system 100 includes an implantable neurostimulator 105 and a lead assembly 110. The implantable neurostimulator 105 may include a housing 115, a feedthrough assembly 120, a power source 125, an antenna 130, and an electronics module 135 (e.g., a computing system). The housing 115 may be comprised of materials that are biocompatible such as bioceramics or bioglasses for radio frequency transparency, or metals such as titanium. In accordance with some aspects of the present invention, the size and shape of the housing 115 may be selected such that the neurostimulator 105 can be implanted within a patient. In the example shown in FIG. 1, the feedthrough assembly 120 is attached to a hole in a surface of the housing 115 such that the housing 115 is hermetically sealed. The feedthrough assembly 120 may include one or more feedthroughs (i.e., electrically conductive elements, pins, wires, tabs, pads, etc.) mounted within and extending through the surface of the housing 115 or a cap from an interior to an exterior of the housing 115. The power source 125 may be within the housing 115 and connected (e.g., electrically connected) to the electronics module 135 to power and operate the components of the electronics module 135. The antenna 130 may be connected (e.g., electrically connected) to the electronics module 135 for wireless communication with external devices via, for example, radiofrequency (RF) telemetry.

In some embodiments, the electronics module 135 may be connected (e.g., electrically connected) to interior ends of the feedthrough assembly 120 such that the electronics module 135 is able to apply a signal or electrical current to conductive traces of the lead assembly 110 connected to exterior ends of the feedthrough assembly 120. The electronics module 135 may include discrete and/or integrated electronic circuit components that implement analog and/or digital circuits capable of producing the functions attributed to the neuromodulation devices or systems such as applying or delivering neural stimulation to a patient. In various embodiments, the electronics module 135 may include software and/or electronic circuit components such as a pulse generator 140 that generates a signal to deliver a voltage, current, optical, or ultrasonic stimulation to a nerve or artery/nerve plexus via electrodes, a controller 145 that determines or senses electrical activity and physiological responses via the electrodes and sensors, controls stimulation parameters of the pulse generator 140 (e.g., control stimulation parameters based on feedback from the physiological responses), and/or causes delivery of the stimulation via the pulse generator 140 and electrodes, and a memory 150 with program instructions operable on by the pulse generator 140 and the controller 145 to perform one or more processes for applying or delivering neural stimulation.

In various embodiments, the lead assembly 110 is a monolithic structure that includes a cable or lead body 155. In some embodiments, the lead assembly 110 further includes one or more thin-film neural interfaces 160 (e.g., an electrode assembly) having one or more electrodes 165, and optionally one or more sensors. In some embodiments, the lead assembly 110 further includes a connector 170. In certain embodiments, the connector 170 is bonding material that bonds conductor material of the cable 155 to the electronics module 135 of the implantable neurostimulator 105 via the feedthrough assembly 120. The bonding material may be a conductive epoxy or a metallic solder or weld such as platinum. In other embodiments, the connector 170 is conductive wire, conductive traces, or bond pads (e.g., a wire, trace, or bond pads formed of a conductive material such as copper, silver, or gold) formed on a substrate and bonds a conductor of the cable 155 to the electronics module 135 of the implantable neurostimulator 105. In alternative embodiments, the implantable neurostimulator 105 and the cable 155 are designed to connect with one another via a mechanical connector 170 such as a pin and sleeve connector, snap and lock connector, flexible printed circuit connectors, or other means known to those of ordinary skill in the art.

The cable 155 may include one or more conductive traces 175 formed on a supporting structure 180. The one or more conductive traces 175 allow for electrical coupling of the electronics module 135 to the electrodes 165 and/or sensors of the thin-film neural interface 160. The supporting structure 180 may be formed with a dielectric material such as a polymer having suitable dielectric, flexibility and biocompatibility characteristics. Polyurethane, polycarbonate, silicone, polyethylene, fluoropolymer and/or other medical polymers, copolymers and combinations or blends may be used. The conductive material for the traces 175 may be any suitable conductor such as stainless steel, silver, copper or other conductive materials, which may have separate coatings or sheathing for anticorrosive, insulative and/or protective reasons.

The thin-film neural interface 160 may include the electrodes 165 and/or sensors fabricated using various shapes and patterns to create certain types of interfaces (e.g., book electrodes, split cuff electrodes, spiral cuff electrodes, epidural electrodes, helical electrodes, probe electrodes, linear electrodes, neural probe, paddle electrodes, intraneural electrodes, etc.). In various embodiments, the thin-film neural interface 160 include a base material that provides support for microelectronic structures including the electrodes 165, a wiring layer, optional contacts, etc. In some embodiments, the base material is the supporting structure 180. The wiring layer may be embedded within or located on a surface of the supporting structure 180. The wiring layer may be used to electrically connect the electrodes 165 with the one or more conductive traces 175 directly or indirectly via a lead conductor. The term “directly”, as used herein, may be defined as being without something in between. The term “indirectly”, as used herein, may be defined as having something in between. In some embodiments, the electrodes 165 may make electrical contact with the wiring layer by using the contacts.

### III. Thin-Film Neural Interfaces

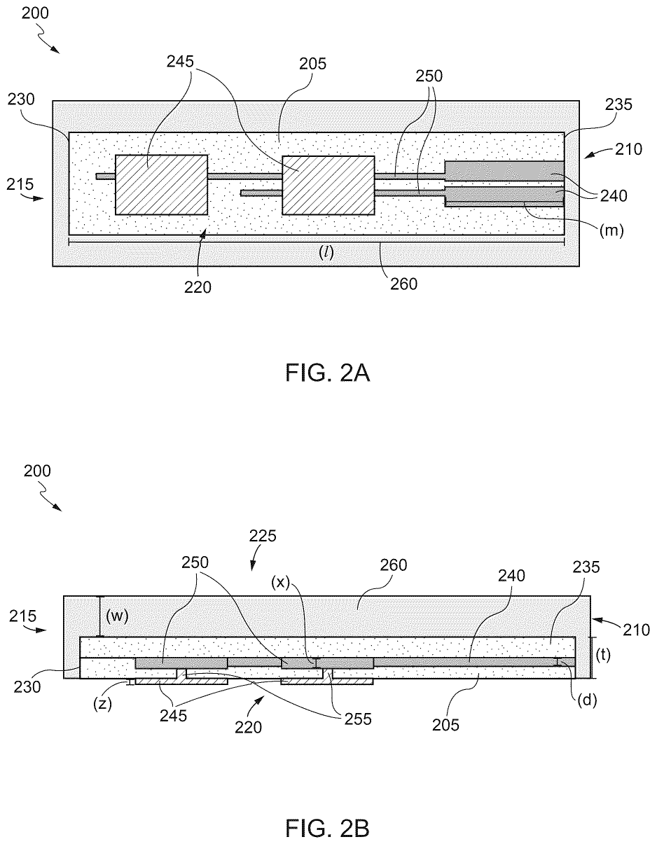

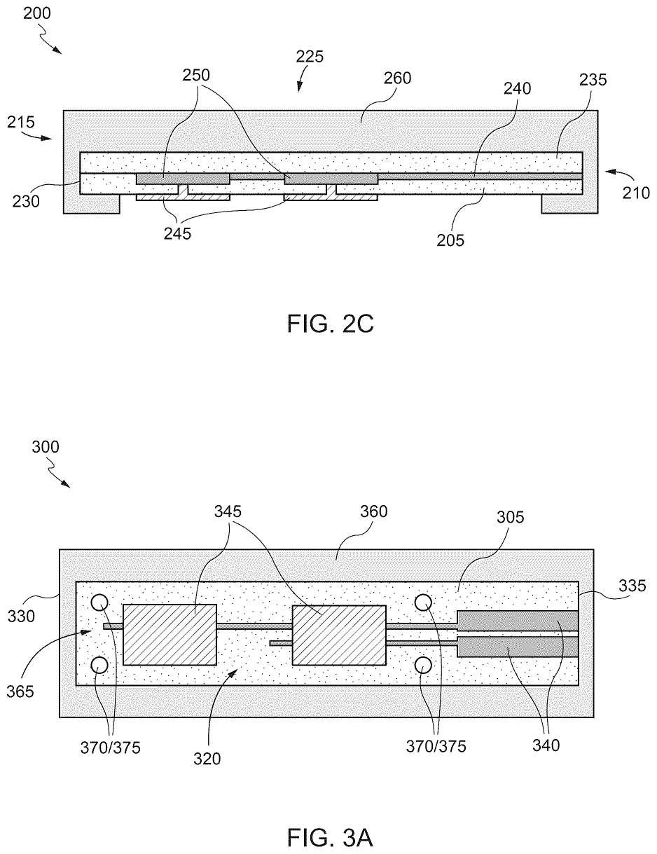

FIGS. 2A and 2B show a thin-film neural interface 200 (e.g., the neural interface 160 described with respect to FIG. 1) in accordance with aspects of the present disclosure. In various embodiments, the neural interface 200 comprises a supporting structure 205 having a proximal end 210 and a distal end 215. As used herein, the term “proximal end” refers to a first end of the supporting structure, while the term “distal end” refers to a second end opposing the first end. For example, the proximal end may be an end of the main body, which is closest to the user, and the distal end may be an end of the main body, which is furthest from the user. The supporting structure 205 may further comprise a front side 220, a back side 225, and opposing edges 230, 235. Although, the opposing edges 230, 235 are shown in the figures as including the edges at the proximal end 210 and the distal end 215 of the supporting structure 205, it should be understood that the opposing edges could additionally or alternatively include the edges on the lateral sides of the supporting structure 205. In various embodiments, the supporting structure 205 of the neural interface and a supporting structure of the cable of the lead assembly are the same structure (i.e., the supporting structure is continuous), which thus creates a monolithic lead assembly. As used herein, the phrase “monolithic” refers to a device fabricated using a same layer of base material.

In various embodiments, the supporting structure 205 is made of one or more layers of dielectric material (i.e., an insulator). The dielectric material may be selected from the group of electrically nonconductive materials consisting of organic or inorganic polymers, ceramics, glass, glass-ceramics, polyimide-epoxy, epoxy-fiberglass, and the like. In some embodiments, the dielectric material is a polymer of imide monomers (i.e., a polyimide), a liquid crystal polymer (LCP) such as Kevlar®, parylene, polyether ether ketone (PEEK), or a combination thereof. In other embodiments, the supporting structure 205 is made of one or more layers of dielectric material formed on a substrate. The substrate may be made from any type of metallic or non-metallic material. In some embodiments, the supporting structure 205 comprising the one or more layers of dielectric material, and optionally the substrate, has a thickness (t) from the front side 220 to the back side 225 and a length (1) from the proximal end 210 to the distal end 215. In some embodiments, the thickness (t) is from 1 μm to 250 μm, from 1 μm to 100, or from 10 μm to 150, for example about 50 μm or about 60 μm. In some embodiments, the length (1) is from 0.5 mm to 25 cm or 0.5 mm to 10 cm, e.g., about 2 cm. As used herein, the terms “substantially,” “approximately” and “about” are defined as being largely but not necessarily wholly what is specified (and include wholly what is specified) as understood by one of ordinary skill in the art. In any disclosed embodiment, the term “substantially,” “approximately,” or “about” may be substituted with “within [a percentage] of” what is specified, where the percentage includes 0.1, 1, 5, and 10 percent.

As shown in FIGS. 2A and 2B, in various embodiments, the neural interface 200 further comprises one or more conductive traces 240 formed on a portion of the supporting structure 205. As used herein, the term “formed on” refers to a structure or feature that is formed on a surface of another structure or feature, a structure or feature that is formed within another structure or feature, or a structure or feature that is formed both on and within another structure or feature. In some embodiments, the one or more conductive traces 240 are formed on the one or more layers of dielectric material of the supporting structure 205. In certain embodiments, the one or more conductive traces 240 are a plurality of traces, for example, two or more conductive traces or from two to twenty-four conductive traces. The one or more conductive traces 240 may be comprised of one or more layers of conductive material. The conductive material selected for the one or more conductive traces 240 should have good electrical conductivity and may include pure metals, metal alloys, combinations of metals and dielectrics, and the like. In some embodiments, the conductive material is gold (Au), gold/chromium (Au/Cr), platinum (Pt), platinum/iridium (Pt/Ir), titanium (Ti), gold/titanium (Au/Ti), or any alloy thereof. In some embodiments, it is also desirable that the conductive material selected for the one or more conductive traces 240 have thermal expansion characteristics or a coefficient of thermal expansion (CTE) that is approximately equal to that of CTE of the supporting structure 205. Matching the CTE of components that contact one another is desirable because it eliminates the development of thermal stresses, which may occur during fabrication and the operation of the cable, and thus eliminates a known cause of mechanical failure in the components.

The one or more conductive traces 240 may be deposited onto a surface of the supporting structure 205 by using thin film deposition techniques well known to those skilled in the art such as by sputter deposition, chemical vapor deposition, metal organic chemical vapor deposition, electroplating, electroless plating, and the like. In various embodiments, the thickness of the one or more conductive traces 240 is dependent on the particular impedance desired for conductor, in order to ensure excellent signal integrity (e.g., electrical signal integrity for stimulation or recording). For example, if a conductor having a relatively high impedance is desired, a small thickness of conductive material should be deposited onto the supporting structure 205. If, however, a signal plane having a relatively low impedance is desired, a greater thickness of electrically conductive material should be deposited onto the supporting structure 205. In some embodiments, each of the one or more conductive traces 240 has a thickness (d). In some embodiments, the thickness (d) is from 0.05 μm to 100 μm, from 0.05 μm to 25 μm, or from 0.1 μm to 15 μm, for example about 0.5 μm or about 10 μm. In some embodiments, each of the one or more conductive traces 240 has a length (m) of about 0.1 mm to 25 cm or 0.5 mm to 10 cm, e.g., about 3 mm.

As shown in FIGS. 2A and 2B, in various embodiments, the neural interface 200 further comprises one or more electrodes 245 formed on the front side 220 of the supporting structure 205 in electrical connection with the one or more conductive traces 240. In some embodiments, the one or more electrodes 245 are in direct electrical connection to the one or more conductive traces 240. In other embodiments, the one or more electrodes 245 are in indirect electrical connection to the one or more conductive traces 240. For example, optionally, the neural interface 200 may further comprise a wiring layer 250 that facilitates the electrical connection between the one or more electrodes 245 and the one or more conductive traces 240.

In some embodiments, the wiring layer 250 is comprised of various metals or alloys thereof, for example, gold (Au), gold/chromium (Au/Cr), platinum (Pt), platinum/iridium (Pt/Ir), titanium (Ti), gold/titanium (Au/Ti), or any alloy thereof. The wiring layer 250 may have a thickness (x) of from 0.05 μm to 100 μm, from 0.5 μm to 15 μm, from 0.5 μm to 10 μm, or from 0.5 μm to 5 μm. In some embodiments, a top surface of the wiring layer 250 is coplanar with a top surface of the supporting structure 205. In other embodiments, the wiring layer 250 is embedded within the supporting structure 205. In yet other embodiments, the wiring layer 250 is formed on the top surface of the supporting structure 205, and the top surface of the wiring layer 250 is raised above the top surface of the supporting structure 205.

In some embodiments, the one or more electrodes 245 are comprised of one or more layers of conductive material, and the conductive material is PEDOT (Poly(3,4-ethylenedioxythiophene)), gold (Au), gold/chromium (Au/Cr), platinum (Pt), platinum/iridium (Pt/Ir), titanium (Ti), gold/titanium (Au/Ti), or any alloy thereof. The one or more electrodes 245 may have a thickness (z) of from 0.05 μm to 150 μm, from 0.05 μm to 50 μm, from 0.05 μm to 25 μm, or from 1 μm to 15 μm. The one or more electrodes 245 may be formed directly on the supporting structure 205. Alternatively, the one or more electrodes 245 may be formed indirectly on the supporting structure 205 (e.g., a layer of polymer such as silicone may be formed between the electrodes and the supporting structure). In some embodiments, contact(s) 255 are formed on the supporting structure 205 and provide the electrical connection between the one or more electrodes 245 and one or more conductive traces 240, optionally via the wiring layer 250. The contact(s) 255 may be comprised of conductive material such as gold (Au), gold/chromium (Au/Cr), platinum (Pt), platinum/iridium (Pt/Ir), titanium (Ti), gold/titanium (Au/Ti), or any alloy thereof, for example.

As shown in FIGS. 2A, 2B, and 2C, in various embodiments, the neural interface 200 further comprises a backing 260 formed on the back side 225 of the supporting structure 205. In some embodiments, backing 260 is comprised of a medical grade polymer material. In certain embodiments, the medical grade polymer material is thermosetting or thermoplastic. For example, the medical grade polymer material may be a soft polymer such as silicone, a polymer dispersion such as latex, a chemical vapor deposited poly(p-xylylene) polymer such as parylene, or a polyurethane such as Bionate® Thermoplastic Polycarbonate-urethane (PCU) or CarboSil® Thermoplastic Silicone-Polycarbonate-urethan (TSPCU). In some embodiments, the backing 260 completely encases a portion of the back side 225 of the supporting structure 205 (see, e.g., FIG. 2A). In some embodiments, the backing 260 completely encases at least the entirety of the back side 225 of the supporting structure 205, and the backing wraps around the opposing edges 230, 235 from the back side 225 of the supporting structure 205 (see, e.g., FIG. 2B). In certain embodiments, the backing 260 is formed coplanar with the front side 220 of the supporting structure 205 (see, e.g., FIG. 2B). In other embodiments, the backing 260 completely encases the back side 225 of the supporting structure 205, extends around the opposing edges 230, 235, and partially encases a portion of the front side 220 of the supporting structure 205 (see, e.g., FIG. 2C). The backing 260 may have an average thickness (w) of from 0.5 μm to 500 μm, from 1.0 μm to 250 μm, from 10 μm to 150 μm, or from 20 μm to 100 μm.

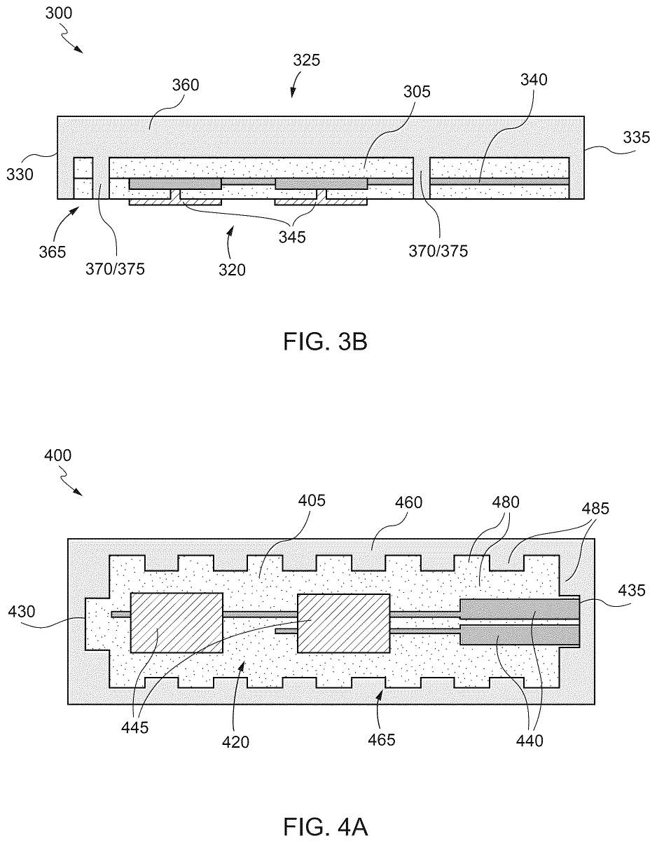

FIGS. 3A and 3B show a thin-film neural interface 300 (e.g., the neural interface 160 or 200 described with respect to FIGS. 1, 2A, 2B, and 2C) in accordance with aspects of the present disclosure. In various embodiments, the thin-film neural interface 300 comprises: (i) a supporting structure 305 comprised of one or more layers of dielectric material, where the supporting structure 305 comprises a front side 320, a back side 325, and opposing edges 330, 335, (ii) one or more electrodes 345 formed on the front side 320 of the supporting structure 305 in electrical connection with one or more conductive traces of a plurality of conductive traces 340, and (iii) a backing 360 (e.g., a medical grade polymer material) formed on the back side 325 of the supporting structure 305. As described with respect to FIGS. 2A, 2B, and 2C, the thin-film neural interface 400 may include additional features such as wiring layers and contacts, which are not repeated here for purposes of brevity.

In some embodiments, the supporting structure 305 includes one or more features 365 for mechanical adhesion with the backing 360. As shown in FIGS. 3A and 3B the one or more features 365 may comprise one or more through holes 370, and the backing 360 fills at least a portion of each of the one or more through holes 370. For example, the backing 360 may comprise rivets 375 formed within the through holes 370 of the supporting structure 305, which in combination, provide additional mechanical adhesion between the supporting structure 305 and the backing 360. The one or more through holes 370 may be formed using conventional lithographic, etching, and cleaning processes, known to those of skill in the art. The number of through holes 370 placed in the supporting structure 305 and corresponding rivets 375 of the backing 360 can be any number depending on the extent of mechanical adhesion and complexity of design desired for the neural interface 300. Moreover, the through holes 370 and respective rivets 375 may be placed in a variety of locations (random or patterned) on the supporting structure 305 and may be a variety of sizes (same or different amongst the plurality of through holes and rivets 375).

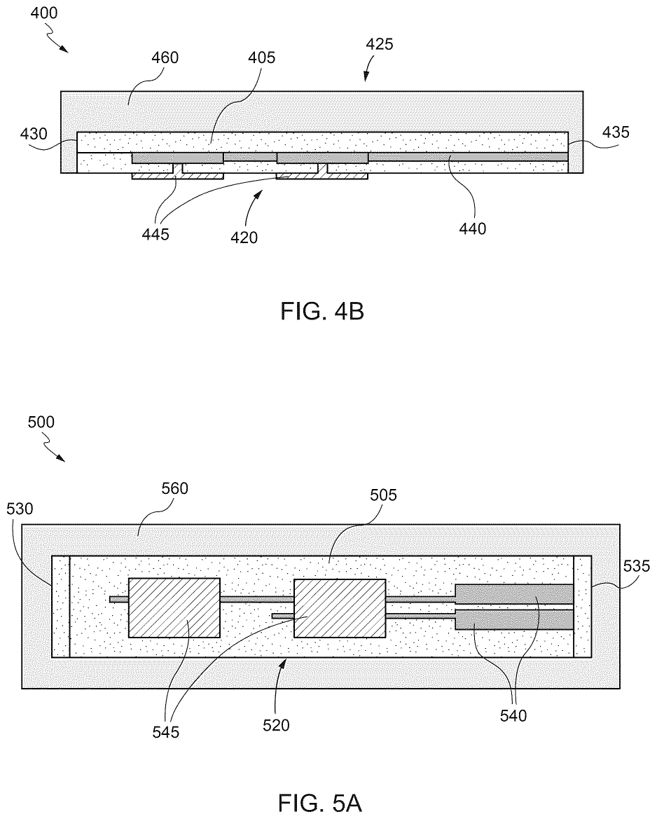

FIGS. 4A and 4B show a thin-film neural interface 400 (e.g., the neural interface 160, 200, or 300 described with respect to FIGS. 1, 2A, 2B, 2C, 3A, and 3B) in accordance with aspects of the present disclosure. In various embodiments, the thin-film neural interface 400 comprises: (i) a supporting structure 405 comprised of one or more layers of dielectric material, where the supporting structure 405 comprises a front side 420, a back side 425, and opposing edges 430, 435, (ii) one or more electrodes 445 formed on the front side 420 of the supporting structure 405 in electrical connection with one or more conductive traces of a plurality of conductive traces 440, and (iii) a backing 460 (e.g., a medical grade polymer material) formed on the back side 425 of the supporting structure 405. As described with respect to FIGS. 2A, 2B, and 2C, the thin-film neural interface 400 may include additional features such as wiring layers and contacts, which are not repeated here for purposes of brevity.

In some embodiments, the supporting structure 405 includes one or more features 465 for mechanical adhesion with the backing 460. As shown in FIGS. 4A and 4B the one or more features 465 may comprise a pattern 480 formed in the one or more layers of dielectric material at the opposing edges 430, 435, and the backing 460 further comprises a pattern 485 in the medical grade polymer material that interlocks with the pattern 480 in the one or more layers of dielectric material. For example, features of the pattern 485 formed in the backing 460 will “lock” (provide additional mechanical adhesion) to features of the pattern 480 formed in the supporting structure 305. The patterns 480, 485 may be formed using conventional lithographic, etching, and cleaning processes (e.g., laser micromachining or reactive ion etching), known to those of skill in the art. The patterns 480, 485 can be any pattern, such as the checkered type pattern shown in the figures or a dovetail type pattern or bow tie type pattern, depending on the extent of mechanical adhesion and complexity of design desired for the neural interface 400.

Moreover, the patterns 480, 485 may be placed in a variety of locations such as all along the contacting edges of the supporting structure and backing or only on certain portions of the edges of the supporting structure and backing and may be a variety of sizes and/or depths.

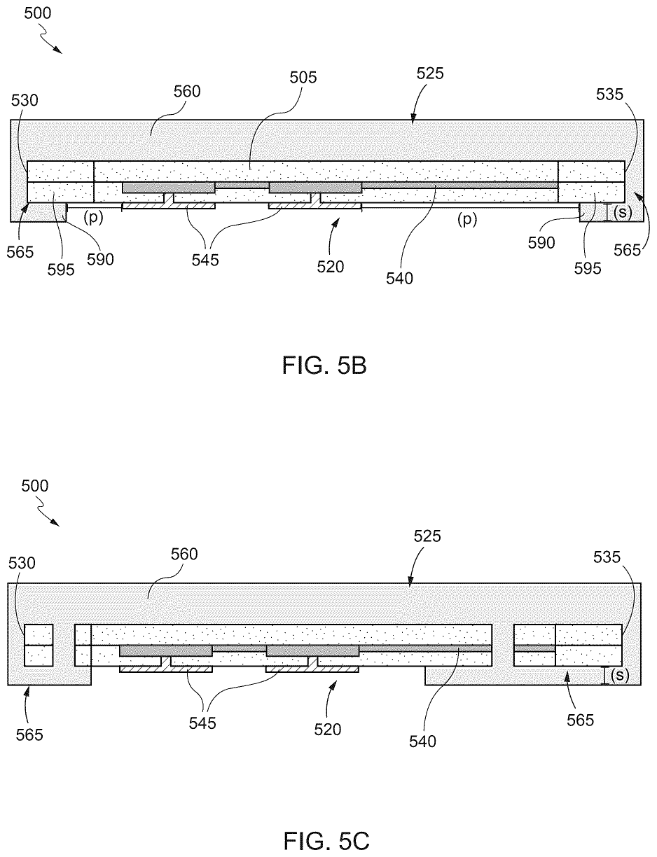

FIGS. 5A and 5B show a thin-film neural interface 500 (e.g., the neural interface 160, 200, 300, or 400 described with respect to FIGS. 1, 2A, 2B, 2C, 3A, 3B, 4A, and 4B) in accordance with aspects of the present disclosure. In various embodiments, the thin-film neural interface 500 comprises: (i) a supporting structure 505 comprised of one or more layers of dielectric material, where the supporting structure 505 comprises a front side 520, a back side 525, and opposing edges 530, 535, (ii) one or more electrodes 545 formed on the front side 520 of the supporting structure 505 in electrical connection with one or more conductive traces of a plurality of conductive traces 540, and (iii) a backing 560 (e.g., a medical grade polymer material) formed on the back side 525 of the supporting structure 505. In some embodiments, the backing 560 is overmolded over the opposing edges 530, 535, and the backing or medical grade polymer material forms a backing layer 590 on a portion of the front side 520 of the supporting structure 505 that is adjacent to the opposing edges 530, 535. As described with respect to FIGS. 2A, 2B, and 2C, the thin-film neural interface 500 may include additional features such as wiring layers and contacts, which are not repeated here for purposes of brevity.

In some embodiments, the supporting structure 505 includes one or more features 565 for mechanical adhesion with the backing 560. As shown in FIGS. 5A, 5B, and 5C the one or more features 565 may comprise the opposing edges 530, 535 being extended or folded 595 to maintain a predetermined distance (p) between the backing layer 590 and the one or more electrodes 545. For example, a layer of backing 590 may be overmolded around the front side 520 of the supporting structure 505 to provide additional mechanical adhesion, and optionally, the edges 530, 535 of the supporting structure 505 may be formed into ‘wings’, or folded so that the backing 560 does not recess the one or more electrodes 545 too far. In some embodiments, the backing layer 590 has a thickness(s) that is less than the thickness (w) of the backing 560. For example, the backing layer 590 may have a thickness(s) from 1.0 μm to 450 μm, from 5.0 μm to 250 μm, from 10 μm to 150 μm, or from 20 μm to 100 μm. The predetermined distance (p) may be from 0.25 mm to 25 mm or from 5 mm to 15 mm, e.g., about 5 mm.

In various embodiments, the one or more features provided to facilitate mechanical adhesion are a single feature (e.g., the through holes, the patterns, or the extensions). In other embodiments, the one or more features provided to facilitate mechanical adhesion are a multiple features (e.g., a combination of two or more of the features: the through holes, the patterns, and the extensions). For example, the through holes may be combined with the patterns, the extensions, or a combination thereof to further facilitate mechanical adhesion. Alternatively, the patterns may be combined with the through holes, the extensions, or a combination thereof to further facilitate mechanical adhesion. Alternatively, the extensions may be combined with the through holes, the patterns, or a combination thereof to further facilitate mechanical adhesion (see, e.g., FIG. 5C, which shows extensions with additional interlocking features (e.g., through holes). In yet other embodiments, the one or more features may be combined with additional features of the backing or other features of the neural interface to further facilitate mechanical adhesion. For example, one or more of the features including the through holes, the patterns, the extensions, or a combination thereof may be combined with the overmold of the backing around the front side of the supporting structure (i.e., the backing layer) to further facilitate mechanical adhesion. Alternatively, one or more of the features including the through holes, the patterns, the extensions, or a combination thereof may be combined with the overmold of the backing around the edges such that the backing is coplanar with the front side of the supporting structure to further facilitate mechanical adhesion. Alternatively, one or more of the features including the through holes, the patterns, the extensions, or a combination thereof may be combined with the overmold of the backing around the front side of the supporting structure over the conductive traces to further facilitate mechanical adhesion.

While the thin-film neural interfaces have been described at some length and with some particularity with respect to a specific design and/or performance need, it is not intended that the thin-film neural interface be limited to any such particular design and/or performance need. Instead, it should be understood the lead assemblies described herein are exemplary embodiments, and that the thin-film neural interface are to be construed with the broadest sense to include variations of the specific design and/or performance need described herein, as well as other variations that are well known to those of skill in the art. In particular, the shape and location of components and layers in the thin-film neural interface may be adjusted or modified to meet specific design and/or performance needs. Furthermore, it is to be understood that other structures have been omitted from the description of the thin-film neural interface for clarity. The omitted structures may include sensor structures, insulating layers, interconnect components, passive devices, etc.

### IV. Methods For Fabricating Neural Interfaces

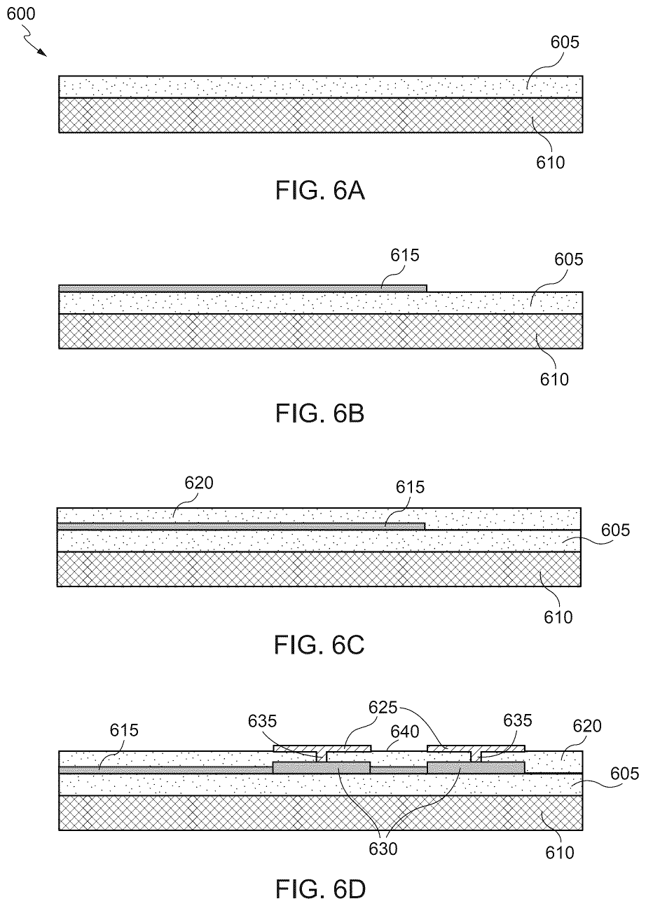

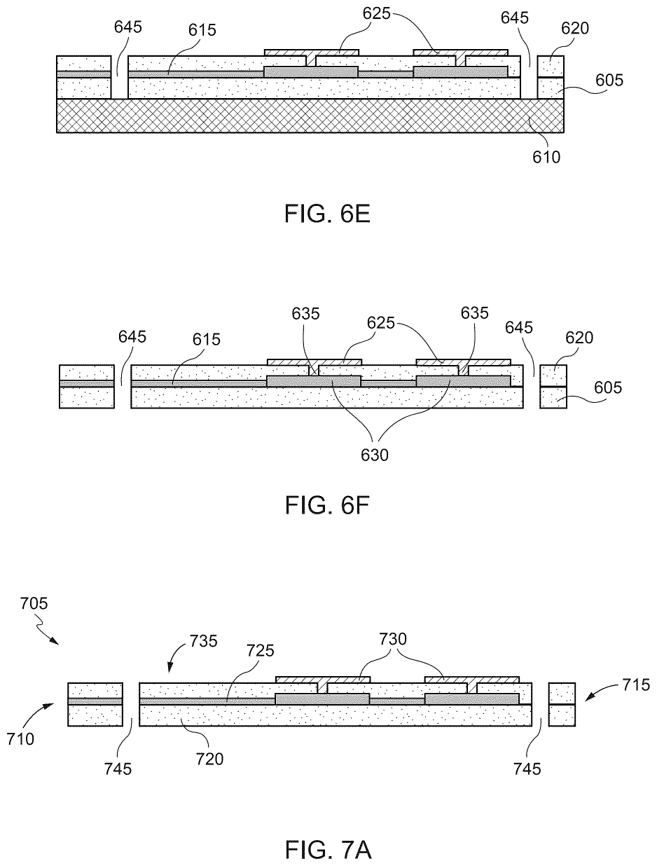

FIGS. 6A-6F show structures and respective processing steps for fabricating a thin-film neural interface 600 (e.g., as described with respect to FIGS. 1, 2A, 2B, 2C, 3A, 3B, 4A, 4B, 5A, or 5B) in accordance with various aspects of the invention. It should be understood by those of skill in the art that the thin-film neural interface can be manufactured in a number of ways using a number of different tools. In general, however, the methodologies and tools used to form the structures of the various embodiments can be adopted from integrated circuit (IC) technology. For example, the structures of the various embodiments, e.g., supporting structure, conductive traces, electrodes, sensors, wiring layers, bond/contact pads, etc., may be built with or without a substrate and realized in films of materials patterned by photolithographic processes. In particular, the fabrication of various structures described herein may typically use three basic building blocks: (i) deposition of films of material on a substrate and/or previous film(s), (ii) applying a patterned mask on top of the film(s) by photolithographic imaging, and (iii) etching the film(s) selectively to the mask.

As used herein, the term “depositing” may include any known or later developed techniques appropriate for the material to be deposited including but not limited to, for example: chemical vapor deposition (CVD), low-pressure CVD (LPCVD), plasma-enhanced CVD (PECVD), semi-atmosphere CVD (SACVD) and high density plasma CVD (HDPCVD), rapid thermal CVD (RTCVD), ultra-high vacuum CVD (UHVCVD), limited reaction processing CVD (LRPCVD), metalorganic CVD (MOCVD), sputtering deposition, ion beam deposition, electron beam deposition, laser assisted deposition, thermal oxidation, thermal nitridation, spin-on methods, physical vapor deposition(PVD), atomic layer deposition (ALD), chemical oxidation, molecular beam epitaxy (MBE), plating (e.g., electroplating), or evaporation.

FIG. 6A shows a beginning structure (a supporting structure) comprising a first polymer layer 605 overlying an optional substrate 610 (e.g., a backer). In various embodiments, the beginning structure may be provided, obtained, or fabricated as a single wafer or panel having a diameter, length, and/or width of less than 15 cm. The substrate 610 may be comprised of any type of metallic or non-metallic material. For example, the substrate 610 may be comprised of but not limited to silicon, germanium, silicon germanium, silicon carbide, and those materials consisting essentially of one or more Group III-V compound semiconductors having a composition defined by the formula AlX1GaX2InX3AsY1PY2NY3SbY4, where X1, X2, X3, Y1, Y2, Y3, and Y4 represent relative proportions, each greater than or equal to zero and X1+X2+X3+Y1+Y2+Y3+Y4=1 (1 being the total relative mole quantity). Substrate 610 may additionally or alternatively be comprised of Group II-VI compound semiconductors having a composition ZnAlCdA2SeB1TeB2, where A1, A2, B1, and B2 are relative proportions each greater than or equal to zero and A1+A2+B1+B2=1 (1 being a total mole quantity). The processes to provide, obtain, or fabricate substrate 610, as illustrated and described, are well known in the art and thus, no further description is provided herein.

The first polymer layer 605 may be comprised of dielectric material (i.e., an insulator). The dielectric material may be selected from the group of electrically nonconductive materials consisting of organic or inorganic polymers, ceramics, glass, glass-ceramics, polyimide-epoxy, epoxy-fiberglass, and the like. In certain embodiments, the dielectric material is a thermoplastic or thermosetting polymer. For example, the polymer may be a polyimide, a LCP, parylene, a PEEK, or combinations thereof. The forming of the first polymer layer 605 may include depositing and curing a dielectric material directly on the substrate 610 without an adhesion promoter. For example, a solution comprised of an imidizable polyamic acid compound dissolved in a vaporizable organic solvent without an adhesion promoter may be deposited (e.g., spin coated) onto the substrate 610. The solution may then be heated at a temperature, preferably less than 250° C., to imidize the polyamic acid compound to form the desired polyimide and vaporize the solvent. The first polymer layer 605 may then be thinned to a desired thickness by planarization, grinding, wet etch, dry etch, oxidation followed by oxide etch, or any combination thereof. This process can be repeated to achieve a desired thickness for the first polymer layer 605. In some embodiments, the first polymer layer 605 may have a thickness from 10 μm to 150 μm. In some embodiments, the first polymer layer 605 may have a thickness from 25 μm to 100 μm. In some embodiments, the first polymer layer 605 may have a thickness from 35 μm to 75 μm.

FIG. 6B shows conductive traces 615 formed in a pattern on a first portion (e.g., region) of the first polymer layer 605. In some embodiments, forming the conductive traces 615 may include depositing a seed layer (e.g., a gold (Au) seed layer, a gold/chromium (Au/Cr) seed layer, platinum (Pt) seed layer, platinum/iridium (Pt/Ir) seed layer, etc.) over the first polymer layer 605. The seed layer may be configured to enable forming of a conductive trace on the first polymer layer 605 (e.g., through Au electroplating, Sn electroplating, Au/Cr electroplating, platinum (Pt) electroplating, platinum/iridium (Pt/Ir) electroplating, etc.). Optionally, and prior to forming of the seed layer, an adhesion layer may be deposited over the first polymer layer 605 to enable adequate application of the seed layer. Deposition of either or both of the adhesion layer and seed layer may include sputter deposition

Following deposition of the seed layer, a resist pattern may be formed above the first polymer layer 605. The resist pattern may include openings that align over at least a portion of the first polymer layer 605 for forming of a plurality of conductive traces 615 (e.g., a conductive layer with a cross-sectional thickness of 0.05 μm to 25 μm or from 0.5 μm to 15 μm) on the first polymer layer 805. For example, the resist may be patterned with openings to form: (i) a first conductive trace 615 over a first region 617 of the first polymer layer 605, and (ii) a second conductive trace 615 over a second region 618 of the first polymer layer 605. It should be understood by those of skill in the art that different patterns and shapes are also contemplated by the present invention based on the design and complexity of the neural interface 600.

In various embodiments, the conductive traces 615 may be deposited through electroplating (e.g., through Au electroplating, Sn electroplating, Au/Cr electroplating, etc.) and may be positioned over at least a portion of the first polymer layer 605 (e.g., the first region 617 and the second region 618). The electroplating maybe performed at a current density of about 4.0 mA/cm2 to about 4.5 mA/cm2. In some embodiments, the exposed area or portion of the first polymer layer 605 may encompass about 8 cm² to about 10 cm². The current may be about 14 mA to about 18 mA and the duration may be from about 110 minutes to about 135 minutes to form the conductive traces 615 having a thickness of about 8 μm to about 10 μm. In other embodiments, the exposed area or portion of the first polymer layer 605 may encompass about 10 cm² to about 18 cm² . The current may be about 18 mA to about 28 mA and the duration may be from about 35 minutes to about 50 minutes to form the wiring layer 615 having a thickness of about 2 μm to about 5 μm.

Following the deposition of the conductive traces 615, the intermediate structure may be subjected to a strip resist to remove the resist pattern and expose portions of the seed layer (portions without wire formation), and optionally the adhesion layer. The exposed portions of the seed layer, and optionally the adhesion layer, may then be subjected to an etch (e.g., wet etch, dry etch, etc.) to remove those portions, thereby isolating the conductive traces 615 over at least a portion of the first polymer layer 605.

FIG. 6C shows an optional second polymer layer 620 formed over the conductive traces 615 and the first portion of the first polymer layer 605. The second polymer layer 620 may be comprised of dielectric material (i.e., an insulator). The dielectric material may be selected from the group of electrically nonconductive materials consisting of organic or inorganic polymers, ceramics, glass, glass-ceramics, polyimide-epoxy, epoxy-fiberglass, and the like. In certain embodiments, the dielectric material is a thermoplastic or thermosetting polymer. For example, the polymer may be a polyimide, a LCP, silicone, parylene, a PEEK, or combinations thereof. The second polymer layer 620 may be comprised of the same material or a different material from that of the first polymer layer 605.

The forming of the second polymer layer 620 may include depositing and curing of a polymer material directly on the conductive traces 615 and the first polymer layer 605. For example, a solution comprised of an imidizable polyamic acid compound dissolved in a vaporizable organic solvent may be applied to the conductive traces 615 and the first polymer layer 605. The solution may then be heated at a temperature, preferably less than 250° C., to imidize the polyamic acid compound to form the desired polyimide and vaporize the solvent. The second polymer layer 620 may then be thinned to a desired thickness by planarization, grinding, wet etch, dry etch, oxidation followed by oxide etch, or any combination thereof. This process can be repeated to achieve a desired thickness for the second polymer layer 620. In some embodiments, the second polymer layer 620 may have a thickness from 1.0 μm to 50.0 μm. In some embodiments, the second polymer layer 620 may have a thickness from 4.0 μm to 15.0 μm. In some embodiments, the second polymer layer 620 may have a thickness from 5.0 μm to 7.0 μm.

In various embodiments, the neural interface 600 may further comprise one or more additional supporting structures that may support one or more additional electronic structures of the interface such as an electrode, sensor, conductor, and/or connector. FIG. 6D shows forming one or more electrodes 625 on the supporting structure 605/610 formed in FIG. 6A that is electrically connected to the conductive traces 615 formed in FIG. 6B. In some embodiments, forming the one or more electrodes 625 comprises forming a wiring layer 630 in a pattern on a second portion of the first polymer layer 605. The wiring layer 630 may be formed at the same time as forming the conductive traces 615, or may be formed subsequent to forming the conductive traces 615. For example, the wiring layer 630 and the conductive traces 615 may be deposited as a continuous layer of conductive material, or may be deposited as two separate metallization layers of conductive material that are in electrical contact with one another. The wiring layer 630 may be formed in the same manner as described in detail with respect to the conductive traces 615.

In some embodiments, forming the one or more electrodes 625 comprises forming the second polymer layer 620 over the wiring layer 630 and the second portion of the first polymer layer 605. As described herein, the second polymer layer 620 may be comprised of dielectric material (i.e., an insulator) selected from the group of electrically nonconductive materials consisting of organic or inorganic polymers, ceramics, glass, glass-ceramics, polyimide-epoxy, epoxy-fiberglass, and the like. In certain embodiments, the dielectric material is a thermoplastic or thermosetting polymer. For example, the polymer may be a polyimide, a LCP, parylene, silicone, a PEEK, or combinations thereof. The second polymer layer 620 may be comprised of the same material or a different material from that of the first polymer layer 605.

In some embodiments, forming the one or more electrodes 630 further comprises forming contact vias 635 in the second polymer layer 620 to the wiring layer 630. The contact vias can e.g. be formed using conventional lithographic, etching, and cleaning processes, known to those of skill in the art. FIG. 6D shows electrodes (optionally one or more sensors) 630 and contacts 635 formed on and within the contact vias 635 to the portion of the top surface the conductive traces 615. In various embodiments, the electrodes 630 (optionally one or more sensors) and contacts 635 may be formed using conventional processes. For example, a conductive material may be blanket deposited on the second polymer layer 620, including within the contact vias 635 and in contact with the portion of the top surface the wiring layer 630. The conductive material may be gold (Au), gold/chromium (Au/Cr), platinum (Pt), platinum/iridium (Pt/Ir), titanium (Ti), gold/titanium (Au/Ti), or any alloy thereof, for example. Once the conductive material is deposited, the conductive material may be patterned using conventional lithography and etching processes to form at least one electrode 625 or a pattern of electrodes 625 as shown in FIG. 6D, for example. In some embodiments, at least one electrode 625 is formed on the second polymer layer 620 such that the at least one electrode 625 is in electrical contact with at least a portion of a top surface of the wiring layer 630. In some embodiments, the pattern of electrodes 625 may include each electrode 625 spaced apart from one another via a portion or region 640 of the second polymer layer 620. It should be understood by those of skill in the art that different patterns are also contemplated by the present invention.

In various embodiments, the neural interface 600 may further comprise one or more features provided to facilitate mechanical adhesion (e.g., the through holes, the patterns, and/or the extensions). FIG. 6E shows forming the one or more features 645 in the supporting structure 605/610/620 formed in FIGS. 6A-6D. In various embodiments, the one or more features 645 are formed in the first polymer layer 605, the second polymer layer 620, or a combination thereof. The one or more features 645 may be formed using conventional lithographic, etching, and cleaning processes, known to those of skill in the art. In various embodiments, the one or more features 645 are a single feature (e.g., the through holes, the patterns, or the extensions). In other embodiments, the one or more features 645 are a multiple features (e.g., a combination of two or more of the features: the through holes, the patterns, and the extensions). For example, the through holes may be combined with the patterns, the extensions, or a combination thereof to further facilitate mechanical adhesion. Alternatively, the patterns may be combined with the through holes, the extensions, or a combination thereof to further facilitate mechanical adhesion. Alternatively, the extensions may be combined with the through holes, the patterns, or a combination thereof to further facilitate mechanical adhesion. In yet other embodiments, the one or more features 645 may be combined with additional features of the backing or other features of the neural interface to further facilitate mechanical adhesion. For example, one or more of the features 645 including the through holes, the patterns, the extensions, or a combination thereof may be combined with the overmold of the backing around the front side of the supporting structure (i.e., the backing layer) to further facilitate mechanical adhesion. Alternatively, one or more of the features 645 including the through holes, the patterns, the extensions, or a combination thereof may be combined with the overmold of the backing around the edges such that the backing is coplanar with the front side of the supporting structure to further facilitate mechanical adhesion. Alternatively, one or more of the features 645 including the through holes, the patterns, the extensions, or a combination thereof may be combined with the overmold of the backing around the front side of the supporting structure over the conductive traces to further facilitate mechanical adhesion.

FIG. 6F shows the thin-film neural interface 600 including the first polymer layer 605, the conductive traces 615, the wiring layer 630, the second polymer layer 620, the electrodes 625, the contacts 635, and the one or more features 645 detached from the substrate 610. In some embodiments, detaching the thin-film lead assembly 600 from the substrate 610 may include laser cutting as final shape (e.g., an elongated rectangle) of the neural interface out of the intermediate structure 645, removal of the substrate (e.g., selective etching), and cleaning (e.g., a step-wise rinsing process) at least top surfaces of the electrodes 630 and the second polymer layer 620 with acetone, isopropyl alcohol, non-ionic surfactant, a liquid detergent system, and/or deionized water to remove residual material such as remaining adhesive material.

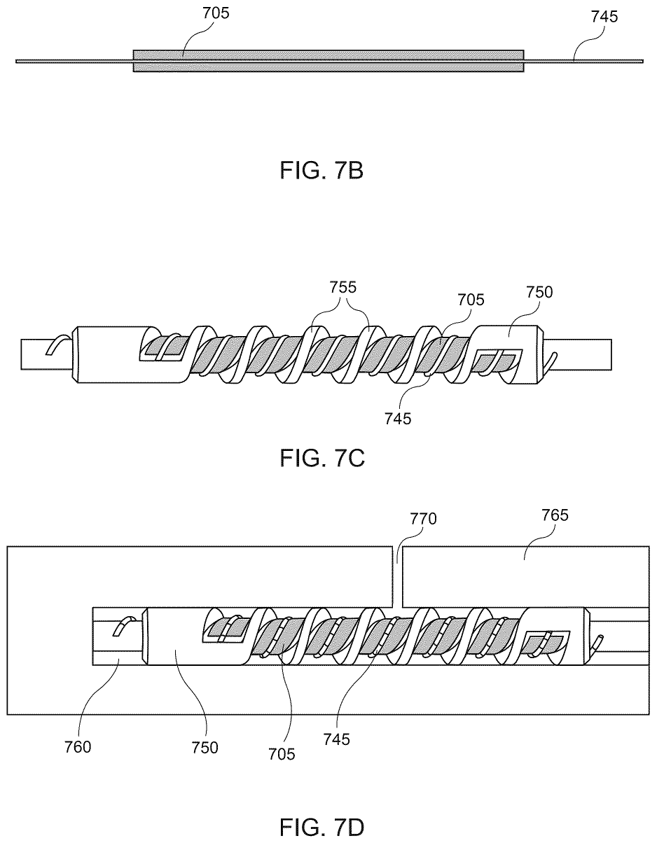

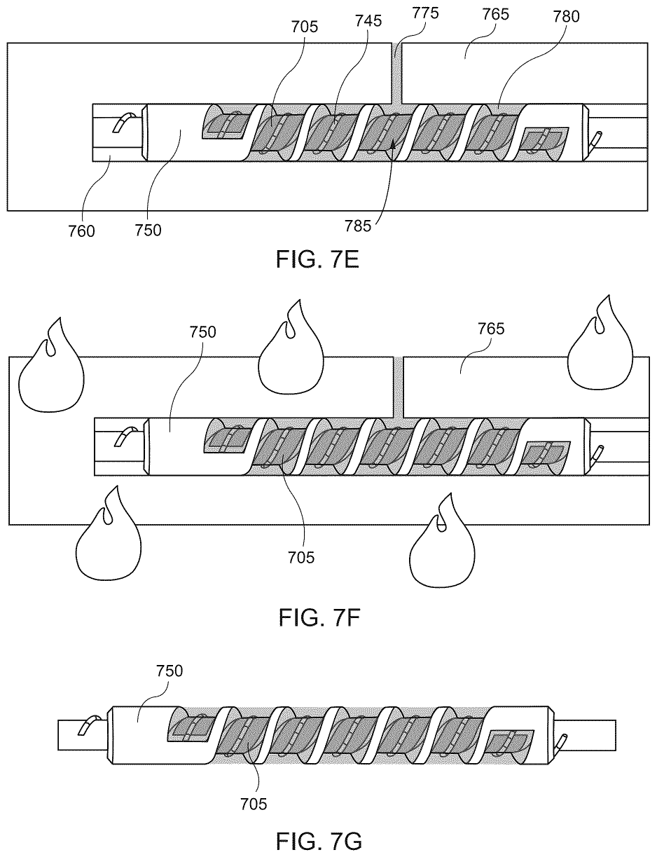

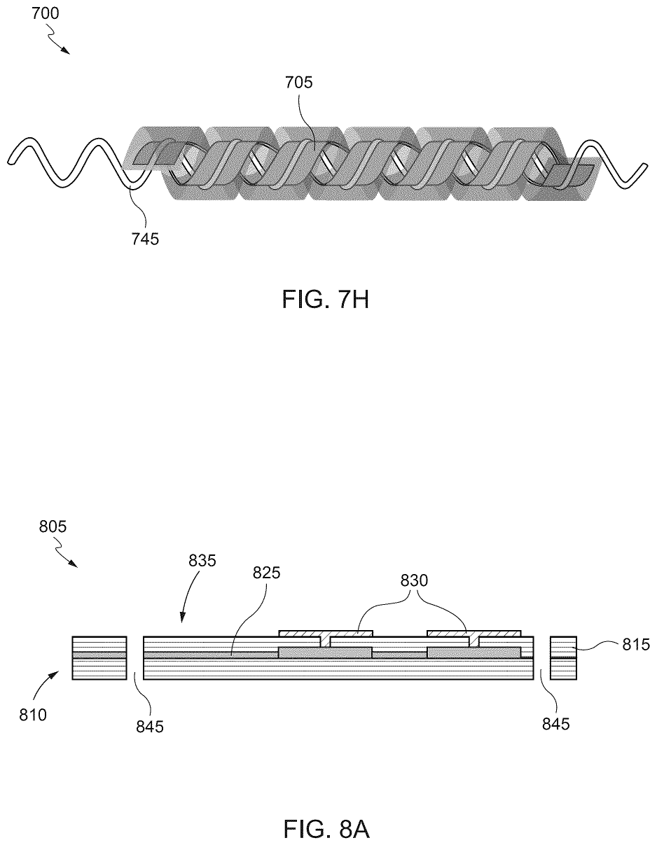

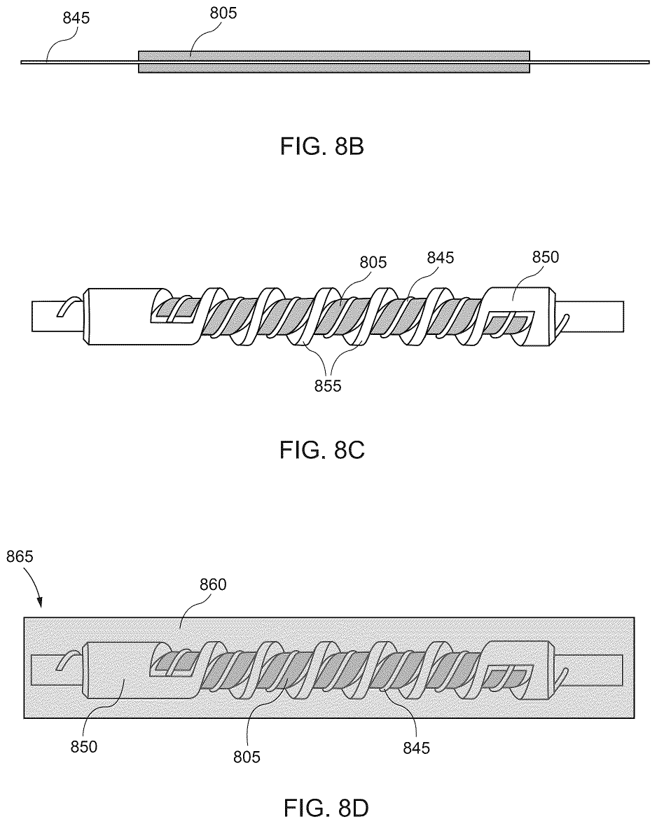

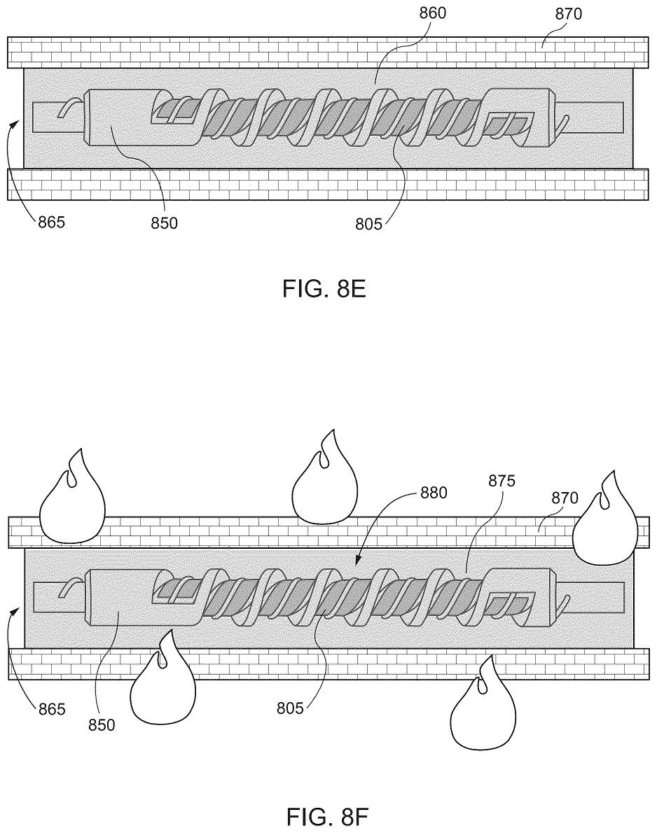

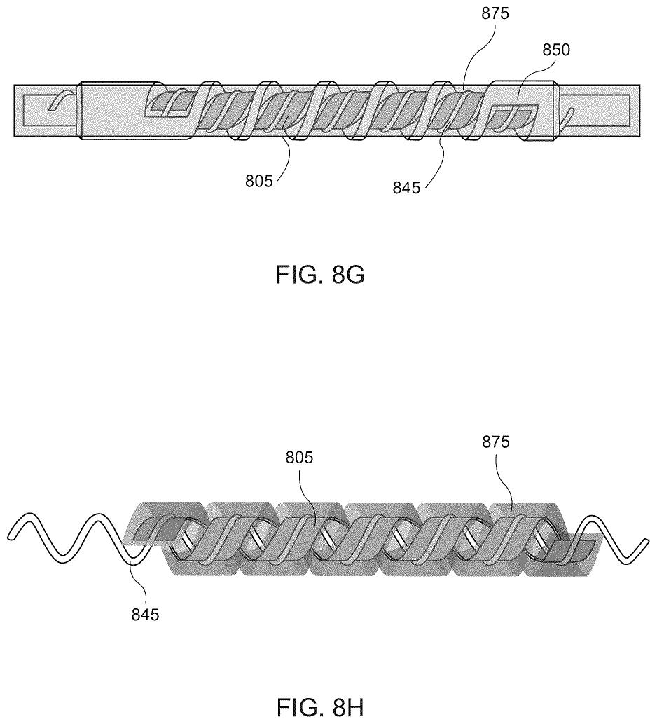

FIGS. 7A-7H show structures and respective processing steps for fabricating a thin-film neural interface 700 (e.g., a neural interface having a soft backing) in accordance with various aspects of the invention. FIG. 7A shows an initial structure 705 for the neural interface 700, the initial structure 705 comprising: a proximal end 710, a distal end 715, a supporting structure 720 that extends from the proximal end 710 to the distal end 715, one or more of conductive traces 725 formed on a portion of the supporting structure 720, and one or more electrodes 730 in electrical connection with the one or more conductive traces of the plurality of conductive traces 725. The one or more electrodes 730 are formed on a front side 735 of the supporting structure 720, and the supporting structure 720 comprises one or more features 740 for mechanical adhesion with a backing. The initial structure 705 may be formed in accordance with the processes describe herein with reference to FIGS. 6A-6F. For example, the initial structure 705 may be laser cut from a wafer or panel fabricated with electroplated traces.

FIG. 7B shows a manipulation device 745 being added to the initial structure 705. In some embodiments, the manipulation device 745 is added to the initial structure 705 using an adhesive to tack the manipulation device 745 to a surface of the initial structure 705. In certain embodiments, the manipulation device 745 is a suture because it is biocompatible and can be chosen to withstand elevated temperatures of molding (typically non-absorbable). In some embodiments, the manipulation device 745 extends from the proximal end to the distal end of the initial structure 705. In certain embodiment the manipulation device 745 hangs over the proximal end, the distal end, or both the proximal and the distal ends.

FIG. 7C shows the initial structure 705 wound (clockwise direction or anti-clockwise direction) into a helical pattern on a mandrel 750. In various embodiments, the mandrel 750 is selected and the winding is controlled such that the neural interface 700 comprises one or more characteristics including a radius, a helix angle, a pitch, a helix length, and a total rise of the helix. For example, a mandrel 750 may be selected with grooves 755 to define the one or more characteristics of the neural interface 700. In some embodiments, the initial structure 705 is wound on the mandrel 750 such that the front side 735 (e.g., the side with one or more electrodes 730) of the initial structure 705 is adjacent to the surface of the mandrel 750. This leaves the back side of the initial structure 705 exposed. In some embodiment, the initial structure 705 is attached to the mandrel 750 using the manipulation device 745. For example, the manipulation device 745 may be routed along ridges or grooves of the mandrel 750 and used to tie the initial structure 705 to the mandrel 750. In some embodiments, the mandrel 750 comprises a coating such as fluorinated ethylene propylene (FEP) or polytetrafluoroethylene (PTFE) for easier removal of the initial structure 705 from the mandrel 750. As should be understood, this is a very flexible process that can accommodate many neural interface shapes or types. For example, the initial structure 705 may be wound on a mandrel 750 (a mandrel having a different geometry than shown in the figures) into other patterns (other than helical) to create different types of neural interfaces, e.g., cuff shaped neural interfaces. Alternatively, the initial structure 705 may be placed into the cavity of a mold without use of a mandrel to create different types of neural interfaces, e.g., planar shaped neural interfaces.

FIG. 7D shows the mandrel 750 with the attached initial structure 705 loaded into a cavity 760 of a mold 765. In some embodiments, the mold 765 comprises the cavity 760 and a gate 770 in fluidic communication with the cavity 760. FIG. 7E shows a backing material 775 (e.g., a solution of silicone) injected into the cavity 760 of the mold 765 via the gate 770 to form a backing 780 over the back side 785 of the supporting structure 720. FIG. 7F shows the backing 780 and the initial structure 705 attached to the mandrel 750 being heated in the mold 765 to form the thin-film neural interface 700 with the backing 780 attached to the back side 785 of the supporting structure 720 via the one or more features 740. The heating process may include baking the mandrel 750 with the attached initial structure 705 in an oven, use of a heat gun, application of hot air, like methods, or any combination thereof. In various embodiments, the mandrel 750 with the attached initial structure 705 are heated at 135° C. to 165° C., for example about 150° C., for 25 to 40 minutes, for example 30 minutes. Thereafter, the mandrel 750 with the attached the attached initial structure 705 in the mold 765 are cooled (e.g., at ambient temperature), the mandrel 750 with the attached initial structure 705 are removed from the mold 765 shown in FIG. 7G, the attached initial structure 705 is withdrawn from the mandrel 750, and the excess backing material, e.g., from the gate, is trimmed off to obtain the final structure of the neural interface 700 shown in FIG. 7H.

Not illustrated in FIGS. 7A-7H but the fabrication process describe therein allows for the selective molding of the backing material 775 onto the supporting structure 720. For example, it may be desirable to selectively mold an extension or tail or backing material 775 that is left exposed for crimping to facilitate connection to the lead body or cable. Furthermore, it should be understood that the manipulation device 745 may be snipped off after molding or left on the neural interface to form a deployment assist feature for the neural interface. Moreover, the mandrel 750 and mold 765 may be textured to assist with release of the backing 780 from the mandrel 750 and mold 765.

FIGS. 8A-8G show structures and respective processing steps for fabricating a thin-film neural interface 800 (e.g., a neural interface having a soft backing) in accordance with various aspects of the invention. FIG. 8A shows an initial structure 805 for the neural interface 800, the initial structure 805 comprising: a proximal end 810, a distal end 815, a supporting structure 820 that extends from the proximal end 810 to the distal end 815, one or more of conductive traces 825 formed on a portion of the supporting structure 820, and one or more electrodes 830 in electrical connection with the one or more conductive traces of the plurality of conductive traces 825. The one or more electrodes 830 are formed on a front side 835 of the supporting structure 820, and the supporting structure 820 comprises one or more features 840 for mechanical adhesion with a backing. The initial structure 805 may be formed in accordance with the processes describe herein with reference to FIGS. 6A-6F. For example, the initial structure 805 may be laser cut from a wafer or panel fabricated with electroplated traces.

FIG. 8B shows a manipulation device 845 being added to the initial structure 805. In some embodiments, the manipulation device 845 is added to the initial structure 805 using an adhesive to tack the manipulation device 845 to a surface of the initial structure 805. In certain embodiments, the manipulation device 845 is a suture because it is biocompatible and can be chosen to withstand elevated temperatures of molding (typically non-absorbable). In some embodiments, the manipulation device 845 extends from the proximal end to the distal end of the initial structure 805. In certain embodiment the manipulation device 845 hangs over the proximal end, the distal end, or both the proximal and the distal ends.

FIG. 8C shows the initial structure 805 wound (clockwise direction or anti-clockwise direction) into a helical pattern on a mandrel 850. In various embodiments, the mandrel 850 is selected and the winding is controlled such that the neural interface 800 comprises one or more characteristics including a radius, a helix angle, a pitch, a helix length, and a total rise of the helix. For example, a mandrel 850 may be selected with grooves 855 to define the one or more characteristics of the neural interface 800. In some embodiments, the initial structure 805 is wound on the mandrel 850 such that the front side 835 (e.g., the side with one or more electrodes 830) of the initial structure 805 is adjacent to the surface of the mandrel 850. This leaves the back side of the initial structure 805 exposed. In some embodiment, the initial structure 805 is attached to the mandrel 850 using the manipulation device 845. For example, the manipulation device 845 may be routed along ridges or grooves of the mandrel 850 and used to tie the initial structure 805 to the mandrel 850. In some embodiments, the mandrel 850 comprises a coating such as FEP or PTFE for easier removal of the initial structure 805 from the mandrel 850. As should be understood, this is a very flexible process that can accommodate many neural interface shapes or types. For example, the initial structure 805 may be wound on a mandrel 850 (a mandrel having a different geometry than shown in the figures) into other patterns (other than helical) to create different types of neural interfaces, e.g., cuff shaped neural interfaces. Alternatively, the initial structure 805 may be placed into the cavity of a mold without use of a mandrel to create different types of neural interfaces, e.g., planar shaped neural interfaces.

FIG. 8D shows the mandrel 850 with the attached initial structure 805 inserted into a polymer tube 860 of backing material to form an intermediate structure 865. In some embodiments, the polymer tube is polyurethane or other thermoplastic materials that may be incorporated as a backing onto the supporting structure 820. Polyurethanes and similar thermoplastic materials are known as an excellent long term implantable insulating material similar to silicone and offer more versatile processing options than silicone such as reflow and are expected to hold shape for longer shelf life than silicone. Thus, use of the polymer tube 860 in some embodiments may allow for a tight cuff geometry or helical geometry that will remain in shape for extended periods of time in a package or during implant as compared to a similar cuff geometry or helical geometry made with a silicone backing. The polymer tube 860 may be melted over the supporting structure, which are formed of polymer materials that have a higher melting temperature than polyurethane or similar thermoplastic materials.

FIG. 8E shows the intermediate structure 865 loaded into a reflow tower or cavity 870. FIG. 8F shows the intermediate structure 865 being heated in reflow tower or cavity 870 to reflow the polymer tube 860 of backing material and form the thin-film neural interface 800 with a backing 875 attached to the back side 880 of the supporting structure 820 via the one or more features 840. The heating process may include baking the intermediate structure 865 in an oven, use of a heat gun, application of hot air, like methods, or any combination thereof. In various embodiments, the intermediate structure 865 is heated at 135° C. to 165° C., for example about 150° C., for 25 to 40 minutes, for example 30 minutes. Thereafter, the intermediate structure 865 and mandrel 850 are removed from the reflow tower or cavity 870 and cooled (e.g., at ambient temperature) shown in FIG. 8G, the attached intermediate structure 865 is withdrawn from the mandrel 850, and the excess backing material is trimmed off to obtain the final structure of the neural interface 800 shown in FIG. 8H.

Not illustrated in FIGS. 8A-8H but the fabrication process describe therein allows for the selective molding of the backing material from the polymer tube 860 onto the supporting structure 820. For example, it may be desirable to selectively mold an extension or tail or backing material that is left exposed for crimping to facilitate connection to the lead body or cable. Furthermore, it should be understood that the manipulation device 845 may be snipped off after molding or left on the neural interface to form a deployment assist feature for the neural interface. Moreover, the mandrel 850 may be textured to assist with release of the backing 875 from the mandrel 850.

While the manufacturing processes of neural interfaces have been described at some length and with some particularity with respect to a specific steps, it is not intended that the processes be limited to any such particular set of steps. Instead, it should be understood that the manufacturing processes described herein are exemplary embodiments, and that the manufacturing processes are to be construed with the broadest sense to include variations of the steps to meet specific design and/or performance need described herein, as well as other variations that are well known to those of skill in the art. For example, the various intermediate and final structures described may be adjusted or modified with treatments to increase wettability of the thin-film lead assembly or to seal the ends of the lumens to meet specific design and/or performance needs. Furthermore, it is to be understood that other steps have been omitted from the description of the manufacturing processes for simplicity and clarity. The omitted steps may include obtaining or fabricating the polymer tubes, waiting predetermined amounts of time for curing or thermosetting, etc.

While the invention has been described in detail, modifications within the spirit and scope of the invention will be readily apparent to the skilled artisan. It should be understood that aspects of the invention and portions of various embodiments and various features recited above and/or in the appended claims may be combined or interchanged either in whole or in part. In the foregoing descriptions of the various embodiments, those embodiments which refer to another embodiment may be appropriately combined with other embodiments as will be appreciated by the skilled artisan. Furthermore, the skilled artisan will appreciate that the foregoing description is by way of example only, and is not intended to limit the invention.

1. (canceled)

2. A thin-film lead assembly comprising:
a cable comprising a supporting structure and a plurality of conductive traces formed on a portion of the supporting structure, wherein the supporting structure is comprised of one or more layers of dielectric material;
a thin-film neural interface formed on the supporting structure at a distal end of the cable, wherein the thin-film neural interface comprises: (i) one or more electrodes formed on a front side of the supporting structure in electrical connection with one or more conductive traces of the plurality of conductive traces, and (ii) a backing formed on a back side of the supporting structure, wherein the backing is comprised of a medical grade polymer material, and the supporting structure includes one or more features for mechanical adhesion with the backing; and
a connector in electrical connection with the one or more conductive traces of the plurality of conductive traces at a proximal end of the cable.

3. The thin-film lead assembly of claim 2, wherein the supporting structure further comprises opposing edges, the backing wraps around the opposing edges from the back side of the supporting structure, and the backing is coplanar with the front side of the supporting structure.

4. The thin-film lead assembly of claim 2, wherein the supporting structure further comprises opposing edges, and the backing wraps around the opposing edges from the back side of the supporting structure.

5. The thin-film lead assembly of claim 2, wherein the one or more features comprise one or more through holes, and the medical grade polymer material fills at least a portion of each of the one or more through holes.

6. The thin-film lead assembly of claim 4, wherein the one or more features comprise a pattern formed in the one or more layers of dielectric material at the opposing edges, and the backing further comprises a pattern in the medical grade polymer material that interlocks with the pattern in the one or more layers of dielectric material.

7. The thin-film lead assembly of claim 4, wherein the backing is overmolded over the opposing edges, the medical grade polymer material forms a backing layer on a portion of the front side of the supporting structure that is adjacent to the opposing edges, and the one or more features comprise the opposing edges being extended or folded to maintain a predetermined distance between the backing layer and the one or more electrodes.

8. The thin-film lead assembly of claim 7, wherein the predetermined distance is from 0.25 mm to 25 mm.

9. A method of manufacturing a thin-film neural interface, comprising:
obtaining an initial structure comprising: a proximal end, a distal end, a supporting structure that extends from the proximal end to the distal end, a plurality of conductive traces formed on a portion of the supporting structure, and one or more electrodes in electrical connection with the one or more conductive traces of the plurality of conductive traces, wherein the one or more electrodes are formed on a front side of the supporting structure, and the supporting structure comprises one or more features for mechanical adhesion with a backing;
adding a manipulation device to the initial structure, wherein the manipulation device extends from the proximal end to the distal end of the initial structure, and the manipulation device hangs over each of the proximal end and the distal end;
attaching, using the manipulation device, the initial structure to a mandrel;
loading the mandrel with the attached initial structure into a cavity of a mold;
injecting a backing material into the cavity of the mold to form a backing over a back side of the supporting structure;
heating the backing and the initial structure attached to the mandrel to form the thin-film neural interface with the backing attached to the back side of the supporting structure via the one or more features; and
removing the mandrel from the thin-film neural interface.

10. The method of claim 9, wherein the obtaining the initial structure comprises:
forming a first polymer layer of the supporting structure on a wafer or panel of substrate;
forming the one or more conductive traces on a first portion of the first polymer layer;
forming a wiring layer on a second portion of the first polymer layer, wherein the forming the wiring layer comprises depositing a conductive material in electrical contact with the one or more of conductive traces;
depositing a second polymer layer of the supporting structure on the wiring layer and the second portion of the first polymer layer;
forming the one or more electrodes on the second polymer layer such that the one or more electrodes are in electrical contact with at least a portion of a top surface of the wiring layer;
forming the one or more features in the first polymer layer, the second polymer layer, or a combination thereof;
cutting the initial structure from the first polymer layer and the second polymer layer; and
removing the initial structure from the wafer or panel of substrate.

11. The method of claim 9, wherein the supporting structure is comprised of one or more layers of dielectric material.

12. The method of claim 11, wherein the one or more layers of dielectric material have a thickness from 1 μm to 100 μm, and the dielectric material is polyimide, liquid crystal polymer, parylene, polyether ether ketone, or a combination thereof.

13. The method of claim 9, wherein the one or more conductive traces have a thickness from 0.05 μm to 25 μm, the one or more conductive traces are comprised of one or more layers of conductive material, and the conductive material is gold (Au), gold/chromium (Au/Cr), platinum (Pt), platinum/iridium (Pt/Ir), titanium (Ti), gold/titanium (Au/Ti), or any alloy thereof.

14. The method of claim 9, wherein the one or more electrodes have a thickness from 0.05 μm to 25 μm, the one or more electrodes are comprised of one or more layers of conductive material, and the conductive material is PEDOT (Poly(3,4-ethylenedioxythiophene)), gold (Au), gold/chromium (Au/Cr), platinum (Pt), platinum/iridium (Pt/Ir), titanium (Ti), gold/titanium (Au/Ti), or any alloy thereof.

15. The method of claim 9, wherein the backing has a thickness from 10 μm to 150 μm, and the backing material is silicone, a polymer dispersion, parylene, or a polyurethane.

16. The method of claim 9, wherein the supporting structure comprises opposing edges, and wherein the mold, the mandrel, or a combination thereof comprises a design feature such that when the backing material is injected or reflowed the backing is formed wrapping around the opposing edges from the back side of the supporting structure, and the backing is coplanar with the front side of the supporting structure.

17. The method claim 9, wherein the supporting structure comprises opposing edges, and wherein the mold, the mandrel, or a combination thereof comprises a first design feature such that when the backing material is injected or reflowed the backing is formed wrapping around the opposing edges from the back side of the supporting structure.

18. The method of claim 9, wherein the one or more features comprise one or more through holes, and the backing material fills at least a portion of each of the one or more through holes.

19. The method of claim 17, wherein the one or more features comprise a pattern formed in the supporting structure at the opposing edges, and wherein the mold, the mandrel, or a combination thereof comprises a second design feature such that when the backing material is injected or reflowed the backing is formed comprising a pattern that interlocks with the pattern in the supporting structure.

20. The method of claim 17, wherein the mold, the mandrel, or a combination thereof comprises a third design feature such that when the backing material is injected or reflowed the backing is overmolded over the opposing edges, the backing material forms a backing layer on a portion of the front side of the supporting structure that is adjacent to the opposing edges, and the one or more features comprise the opposing edges being extended or folded to maintain a predetermined distance between the backing layer and the one or more electrodes, wherein the predetermined distance is from 0.25 mm to 25 mm.

21. A method of manufacturing a thin-film neural interface, comprising:
obtaining an initial structure comprising: a proximal end, a distal end, a supporting structure that extends from the proximal end to the distal end, a plurality of conductive traces formed on a portion of the supporting structure, and one or more electrodes in electrical connection with the one or more conductive traces of the plurality of conductive traces, wherein the one or more electrodes are formed on a front side of the supporting structure, and the supporting structure comprises one or more features for mechanical adhesion with a backing;
adding a manipulation device to the initial structure, wherein the manipulation device extends from the proximal end to the distal end of the initial structure, and the manipulation device hangs over each of the proximal end and the distal end;
attaching, using the manipulation device, the initial structure to a mandrel;
inserting the mandrel with the attached initial structure into a tube of backing material to form an intermediate structure;
heating the intermediate structure to reflow the tube of backing material and form the thin-film neural interface with the backing attached to a back side of the supporting structure via the one or more features; and
removing the mandrel from the thin-film neural interface.
